(19) United States
(12) Patent Application Publication
(10) Pub. No.: US 2012/0053083 A1
(43) Pub. Date: Mar. 1, 2012

(54) HEPATOCELLULAR CARCINOMA

(75) Inventors: Anneleen Daemen, Kinrooi (BE); Bart De Moor, Bierbeek (BE); Olivier Gevaert, Kessel-Lo (BE); Louis Libbrecht, Diksmuide (BE); Hannah Van Malenstein, Leuven (BE); Jos Van Pelt, Kessel-Lo (BE); Chris Verslype, Kessel-Lo (BE)

(73) Assignee: KATHOLIEKE UNIVERSITEIT LEUVEN, Leuven (BE)

(21) Appl. No.: 13/318,789

(22) PCT Filed: May 5, 2010
(86) PCT No.: PCT/BE2010/000037

§ 371 Date: Nov. 4, 2011

(30) Foreign Application Priority Data
May 5, 2009 (GB) 0907658.9
Jun. 16, 2009 (GB) 0910278.1
Dec. 7, 2009 (GB) 0921365.3

(51) Int. Cl. C40B 30/04; C12Q 1/68

(52) U.S. Cl. 506/9; 435/6.14; 435/6.12

(57) ABSTRACT

Present invention concerns a kit and an in vitro method, for evaluating a biological stage of an HCC tumour in an individual, based on a sample from the individual, comprising: deriving from the sample a profile data set, the profile data set on a the gene expression panel with the markers CCNG2, EGLN3, ERO1L, FGF21, MAT1A, RCL1 and WDR45L or a substantially similar marker, being a quantitative measure of the amount of a distinct RNA or protein constituent in the panel so that measurement of the constituents enables evaluation of the biological condition or the biological behaviour HCC tumours.

### BACKGROUND OF THE INVENTION

A. Field of the Invention

The present invention relates generally to profiling of the biological condition of a biological sample, more particularly a sample of a hepatocellular carcinoma (HCC) tumour, for identifying the morbidity, stage or behaviour of the HCC, including obtaining the expression profile of cyclin G2 (CCNG2), EGL nine homolog 3 (EGLN3), ERO1-like (S. cerevisiae) (ERO1L), Fibroblast Growth Factor 21 (FGF21), methionine adenosyltransferase 1, alpha (MAT1A), RNA terminal phosphatase cyclase-like 1 (RCL1) and WD repeat domain phosphoinositide-interacting protein 3 (WDR45L) and identifying different patterns of the CCNG2, EGLN3, ERO1L, FGF21, MAT1A, RCL1 and WDR45L gene expression. The present invention thus solves the problems of the related art of deciding on the proper treatment of HCC by identifying from a plurality of genes that are deregulated in HCC, a set of gene or protein markers of which the expression profile correlates to the severity of the HCC and is decisive for the pharmacological or other interventions for HCC.

Several documents are cited throughout the text of this specification. Each of the documents herein (including any manufacturer's specifications, instructions etc.) are hereby incorporated by reference; however, there is no admission that any document cited is indeed prior art of the present invention.

B. Description of the Related Art

Hepatocellular carcinoma (HCC) is the sixth most common malignancy in the world and the third most common cause of cancer related deaths (Parkin 2005). Every year 600,000 new cases are diagnosed and almost just as many patients die annually of this disease (Parkin 2005). The incidence in Western countries is increasing due to the rise in hepatitis C (HCV) and non-alcoholic fatty liver disease (NAFLD). The most important risk factor for the development of HCC is cirrhosis, which is present in 80% of patients. Cirrhosis can be caused by different pathologies, such as hepatitis B (HBV) or hepatitis C virus, alcohol intoxication, haemochromatosis or NAFLD. HCC has become the most common cause of death in patients with cirrhosis in Europe (Fattovich 1997).

Hepatocellular carcinomas (HCCs) are heterogeneous tumours with respect to etiology, cell of origin and biology. The course of the disease is unpredictable and is in part dependent on the tumour microenvironment. To come to objective prognostic criteria to decide on treatment options several research groups have tried to identify HCC-specific and predictive gene signatures, but unfortunately in each of these studies the gene signature was not generally applicable but limited to and only valid for the study it originated from. All these microarray studies show remarkably little overlap and it is difficult to find a clear correlation between the molecular classes and prognosis. Major obstacles are the limited number of patients and variable underlying etiologies from which both clinical and corresponding molecular data are available. The results of the studies seem to be center dependent because of the different microarray techniques used, the small heterogeneous cohorts that are studied and the different clinical parameters used for the evaluation. There is accordingly a need for general prognostic criteria to diagnose and decide on treatment options and in the treatment of HCCs.

One of the microenvironmental factors is hypoxia, which is known to promote aggressiveness in other malignant tumours. Liver cancer usually develops in a cirrhotic environment where the blood flow is already impaired and more importantly, during the expansion of the tumor the neovascularization is unorganized with leaky blood vessels, arteriovenous shunting, large diffusion distances and coiled vessels. These structural and functional defects lead to both acute hypoxia due to fluctuating flow and to chronic hypoxia due to diffusion distances of more than 150 μm. We hypothesized that in HCC there are regions with sustained hypoxia that induce a characteristic gene expression pattern. Moreover, during the development of HCC there is an important contribution of this chronic hypoxia on prognosis via this gene expression pattern. Until now, most research has been performed in acute hypoxic models (<24 hours). We identified a 7-gene signature, which is associated with chronic hypoxia and generally predicts prognosis in patients with HCC. In the future this signature could be used as a diagnostic tool. In addition, chronic hypoxia gene expression information can be used in the search for new therapeutic targets.

Thus, the present invention accordingly provides the means to predict the biological behaviour of HCC tumours and the course of the disease in order to decide on the proper treatment by a method of quantifying the expression of a cluster of CCNG2, EGLN3, ERO1L, FGF21, MAT1A, RCL1 and WDR45L genes.

This allows to carry out hepatocellular carcinomas grading or HCC staging. A system and method has been provided for staging or grading the HCC in a biological sample, preferably a tumour bioptic sample of an individual comprising: a) assessing the amount of a CCNG2 mRNA, EGLN3 mRNA, ERO1L mRNA, FGF21 mRNA, MAT1A mRNA, RCL1 mRNA and WDR45L mRNA or assessing the amount of CCNG2, EGLN3, ERO1L, FGF21, MAT1A, RCL1 and WDR45L expressing product in said biological sample and b) comparing the amount of a CCNG2 mRNA, EGLN3 mRNA, ERO1L mRNA, FGF21 mRNA, MAT1A mRNA, RCL1 mRNA and WDR45L mRNA or of CCNG2, EGLN3, ERO1L, FGF21, MAT1A, RCL1 and WDR45L expressing product for each of the mRNA or the expression products with predetermined standard values that are indicative of a risk of mortality of HCC or indicative for the behaviour of the HCC tumour or for the treatment of the HCC.

More particularly this allows carrying out hepatocellular carcinomas grading or HCC staging. A system and method has been provided for staging or grading the HCC in a biological sample, preferably a tumour bioptic sample of an individual comprising: a) assessing the amount of a CCNG2 mRNA, EGLN3 mRNA, ERO1L mRNA, FGF21 mRNA, MAT1A mRNA, RCL1 mRNA and WDR45L mRNA or assessing the amount of CCNG2, EGLN3, ERO1L, FGF21, MAT1A, RCL1 and WDR45L expressing product in said biological sample and b) comparing the ratio value for each of the mRNA or the expression products to at least one predetermined cut-off value, wherein a ratio value above said predetermined cut-off value is indicative of a risk of mortality of HCC or indicative for the behaviour of the HCC tumour or for the treatment of the HCC or its use to decide on the proper treatment or proper medicament of the HCC disease state.

The invention moreover provides a method for differentiating between HCC subtypes in a patient comprising a) determining an amount of a CCNG2, EGLN3, ERO1L, FGF21, MAT1A, RCL1 and WDR45L gene expression level in a HCC tumour sample preferably of a HCC biopsy obtained from the individual; and b) correlating the amount of the CCNG2, EGLN3, ERO1L, FGF21, MAT1A, RCL1 and WDR45L gene expression level in the sample with the presence of a HCC subtype in the individual.

### SUMMARY OF THE INVENTION

The present invention solves the problems of the related art of deciding on the proper treatment of HCC.

The present invention identified from a plurality of genes that are deregulated in HCC, a set of gene or protein markers of which the expression profile is correlated to the severity of the HCC and is decisive for the pharmacological or other interventions for HCC.

Present invention demonstrates a unique, liver specific 7-gene signature associated with chronic hypoxia that correlates with poor prognosis in HCCs. An expression of least three genes of this liver specific gene set allows the assessment of the biological behaviour of HCC tumours and the prediction of the survival and recurrence.

In accordance with the purpose of the invention, as embodied and broadly described herein, the invention is broadly drawn to the staging of HCC in a subject and making a decision on a treatment thereto by a biological condition of a HCC sample from an individual. It is based on the characterization of a set of genes (the HCC hypoxia marker genes) which are differentially expressed under chronic hypoxia and whose expression profile is able to predict the prognosis of patients with HCC. It is thus a first aspect of the present invention to provide in vitro methods to determining hypoxia in an HCC tumour and in staging HCC, said methods including the use of a gene expression profile data set having a quantitative measure of the RNA or protein constituents of the group of genes consisting of CCNG2, EGLN3, ERO1L, FGF21, MAT1A, RCL1 and WDR45L.

Within said set of genes a particular subset consists of RCL1, ERO1L and MAT1A. For said genes, it has now been demonstrated that they are functionally linked to hypoxia or a hypoxic response, and that the expression levels of said genes correlate to the severity of HCC. Thus, in a particular embodiment of the invention the staging of HCC is based on the expression profile of RCL1 in combination with one, two, three, four, five or more genes selected from the group consisting of CCNG2, EGLN3, ERO1L, FGF21, MAT1A, and WDR45L; more in particular RCL1 in combination with one, two, three, four or five genes selected from the group consisting of WDR45L, MAT1A, ERO1L, CCNG2 and EGLN3; even more in particular of RCL1 in combination with WDR45L; with MAT1A or with WDR45L and MAT1A.

The present invention concerns a new cluster of correlating molecules of the group consisting of CCNG2, EGLN3, ERO1L, FGF21, MAT1A, RCL1 and WDR45L; including subsets thereof like RCL1, ERO1L and MAT1A, in a tissue or at least one cell of a tissue for instance a cell of a tissue biopsy, preferably a HCC tumour biopsy, and of identifying the condition of the genes expressing said correlating molecules or of the expression levels of said molecules in a method or system for identifying the stage or aggressiveness of such HCC tumour. In said respect, the amount of upregulation, i.e. the amount of increase in expression level of the genes WDR45L, CCNG2, EGLN3 and ERO1L; and the amount of downregulation, i.e. the amount of decrease in expression level of the genes RCL1, MAT1A and FGF21; is indicative for hypoxia in said HCC tumour and accordingly an indication for the severity or invasiveness of said HCC tumour.

This system of method provides information on how to modulate the correlating molecules to treat the HCC. Several options of HCC treatment are available in the art such as liver transplantation, surgical resection, percutaneous ethanol injection (PED, transcatheter arterial chemoembolization (TACE), sealed source radiotherapy, radiofrequency ablation (RFA), Intra-arterial iodine-131-lipiodol administration, combined PEI and TACE, high intensity focused ultrasound (HIFU), hormonal therapy (e.g. Antiestrogen therapy with tamoxifen), high intensity focused ultrasound (HIFU), adjuvant chemotherapy, palliative regimens such as doxorubicin, cisplatin, fluorouracil, interferon, epirubicin, taxol or cryosurgery. It is accordingly a further objective of the present invention to provide the use of the aforementioned methods in determining the biological condition or biological behaviour of an HCC tumour, wherein an increase of hypoxia in said tumour is indicative for an increased severity or invasiveness of said tumour.

It is also an aspect of the present invention to provide kits for use in performing the in vitro methods of the present invention and comprising means for determining the level of gene expression of the cluster(s) of genes described herein, i.e. the group consisting of CCNG2, EGLN3, ERO1L, FGF21, MAT1A, RCL1 and WDR45L; and any subsets thereof like RCL1, ERO1L and MAT1A. As the level of gene expression is either determined at the nucleic acid or the protein level, the means to determine said gene expression typically and respectively consist of one or more oligonucleotides that specifically hybridize to the HCC hypoxia marker genes, or of one or more antibodies that specifically bind to the proteins encoded by the HCC hypoxia marker genes of the present invention.

In overview a particular embodiment 1 of present can be an in vitro method for determining the biological behaviour of a HCC tumour from an individual comprising (a) determining the level of gene expression corresponding to 3, 4, 5, 6, or 7 markers selected among CCNG2, EGLN3, ERO1L, FGF21, MAT1A, RCL1 and WDR45L in a test HCC tumour sample obtained from an individual, to obtain a first set of value, and (b) comparing the first set of value with a second set of value corresponding to the level of gene expression assessed for the same gene(s) and under identical condition as for step a) in a HCC tumour sample with a defined biological behaviour history to define the biological behaviour of said test HCC tumour. Furthermore the invention can comprise

1) The in vitro method of embodiment 1, said method comprising determining the level of gene expression of RCL1 and of 2, 3, 4, or 5 other gene(s) selected from the group consisting of WDR45L, MAT1A, ERO1L, CCNG2 and EGLN3. The in vitro method of embodiment 1, said method comprising determining the level of gene expression of RCL1 and determining the level of gene expression of WDR45L; MAT1A or of WDR45L and MAT1A.

2) The in vitro method of embodiment 1, whereby the amount of upregulation of CCNG2, EGLN3, ERO1L or WDR45L and the amount of downregulation of FGF21, MAT1A or RCL1 is indicative for increased severity or invasiveness of the HCC tumour.

3) The in vitro method of embodiment 1, whereby the amount of upregulation of CCNG2, EGLN3, ERO1L or WDR45L and the amount of downregulation of FGF21, MAT1A or RCL1 is indicative for increased proliferation in the HCC tumour.

4) The in vitro method of embodiment 1, whereby the amount of upregulation of CCNG2, EGLN3, ERO1L or WDR45L and the amount of downregulation of FGF21, MAT1A or RCL1 is indicative for increased morbidity of the HCC tumour.

5) The in vitro method of any one of the previous claims whereby the defined biological behaviour of said tumour is predictive for the chance of recurrence after treatment or tumour removal

6) The in vitro method of any one of the previous claims whereby the defined biological behaviour of said tumour is predictive for survival after treatment or tumor removal.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description. It is to be understood that both the foregoing general description and the following detailed description are exemplary and explanatory only and are not restrictive of the invention, as claimed.

### BRIEF DESCRIPTION OF THE DRAWINGS

The present invention will become more fully understood from the detailed description given herein below and the accompanying drawings which are given by way of illustration only, and thus are not limitative of the present invention, and wherein:

FIG. 1. displays the gene expression in cultures of HepG2 cells after exposure to hypoxia as determined by Quantitative RT-PCR 1A) Hypoxia related genes. HIF1A, HIF1A regulators (EGLN1 and FIH) and HIF1A target gene VEGF were assayed by real time PCR. Expression ratio (log base 2) was determined in parallel cultures with β2M as house keeping gene and expressed as increase (positive) or decrease compared to control cultures kept at 20% O₂. 1B) Top genes from microarray for confirmation. We chose BCL2, CDO1, LOX, ADM and IGFBP from the list of most significant altered genes and determined expression ratio (as described in 1A).

FIG. 2. provides two graphs of the immunohistochemical staining score for (2A) HIF1A and (2B) VEGF after exposure to normal (20%) or impaired (2%) oxygen at several timepoints. To evaluate the staining a semi-quantitative quickscore (1-9) was used which combines positivity (P) with a range from 1-6 and intensity (I), with a range from 0-3. (Detre 1995).There is a strong induction of both proteins in the acute phase (0-24 hours), but after prolonged hypoxia a new balance occurs. HIF1A is not expressed under normal oxygen (20%) conditions, whereas VEGF has a low constitutional expression.

FIG. 3. provides an immunohistochemical staining under hypoxic conditions A) HIF1A staining at 0 hrs—there is no HIF1A present. B) HIF1A staining after 24 hrs—almost all cells are positive. C) HIF1A staining after 72 hrs—some cells are positive. D) VEGF staining after 0 hrs—a single cell shows constitutional expression. E) VEGF staining after 24 hrs—cytoplasm of most cells stains positive. F) VEGF staining after 72 hrs—some cells are positive (A, D: 20% O₂, B,C,E,F: 2% O₂) The arrows indicate cells with positive staining, the number of arrows represents the percentage of staining (see also FIG. 2).

FIG. 4 demonstrates the selection procedure of 7 gene prognostic hypoxia gene set. Starting from the 265 genes that were identified from the microarray experiments with HepG2 cells we followed several steps that led us to identify a 7 gene set that was present in the studies by Wurmbach, Lee en Boyault. The prognostic value was subsequently confirmed when we tested this set on the study of Chiang.

FIG. 5 provides the ROC-curves. 5A. ROC-curves for the three training sets. The AUC for Wurmbach (Vascular invasion)=88.9%, the AUC for Boyault (FAL-index)=72.8% and the AUC for Lee (Clusters)=84.9%. 5B. ROC-curves for the validation set after application of the 7-gene prognostic signature. A division was made between BCLC-stage 0+A+B vs. C. (AUC=91.0%) and a division between BCLC-stage 0+A vs B+C. (AUC=71.5%)

FIG. 6 provides hypoxia scores. 6A Hypoxia score based on the hypoxia 7 gene set applied to the clusters used by Chiang. 6B Hypoxia score based on the hypoxia 7 gene set applied to the clusters used by Boyault

FIG. 7: displays the mRNA expression of the 7 genes in normal human tissues. Expression values were classified in 4 groups: 0=<20% (light grey/dots), 1=20-50% (medium grey), 2=40-70% (black) and 3=>70% (not displayed) as reported in NCBI-data base (in FIG. 7 of this application displayed by a grey scale and number code). The mean for each gene was determined and presented in this table. Blank means that no data are available for that gene in the 4 sets used. MAT1A, FGF21 and RCL1 will be downregulated under hypoxia in HCC and EGLN3, ERO1L, WDR45L and CCNG2 will be upregulated under hypoxia in HCC.

FIG. 8: provides the sequence (SEQ. ID 1) of the Homo sapiens cyclin G2, mRNA (cDNA clone MGC:45275), complete cds with accession BCO32518 (locus BC032518 2074 bp mRNA as deposited on 7 October 2003 (FIG. 8A) and the sequence of the CCNG2 protein that it encodes (SEQ. ID 2). (FIG. 8B) Related nucleotide sequences are the genomic sequences AC 104771.4 (101278 . . . 110697), AF549495.1 and CH471057.1, mRNA sequence AK292029.1, AK293899.1, BC032518.1, BT019503.1, CA429362.1, CR542181.1, CR542200.1, CR593444.1, DC344594.1, L49506.1, U47414.1, DQ890836.2 and DQ893991.2 and the protein sequences AAN40704.1, EAX05812.1, EAX05813.1, EAX05814.1, BAF84718.1, BAG57286.1, AAH32518.1, AAV38310.1, CAG46978.1, CAG46997.1, AAC41978.1 and AAC50689.1 as deposited date 5 April 2009

FIG. 9 provides the sequence (SEQ. ID 3) of the Homo sapiens egl nine homolog 3 (EGLN3), mRNA with accession NM_{—}022073 NM_{—}033344 (locus NM_{—}022073 2722 bp mRNA as deposited on PRI 28 December 2008 (FIG. 9B) and the sequence of the EGLN3 protein (FIG. 9A) that it encodes (SEQ. ID 4). Related nucleotide sequences are the genomic sequences AL358340.6 and CH471078.2, the mRNA sequences AJ310545.1, AK025273.1, AK026918.1, AK123350.1, AK225473.1, BC010992.2, BC064924.1, BC102030.1, BC105938.1, BC105939.1, BC111057.1, BG716229.1, BX346941.2, BX354108.2, CR591195.1, CR592368.1, CR606051.1, CR608810.1, CR611178.1, CR613124.1, CR620175.1, CR623500.1 and DQ975379.1 and the protein sequences, EAW65929.1, CAC42511.1, BAB15101.1, BAG53892.1, AAH10992.3, AAH64924.2, AAI02031.1, AAI05939.1, AAI05940.1 and AAI11058.2 as deposited date 5 April 2009.

FIG. 10: provides the sequence (SEQ. ID 5) of the Homo sapiens ERO1-like (S. cerevisiae) (ERO1L), mRNA with accession NM_{—}014584 (locus NM_{—}014584 3334 bp mRNA as deposited on 21 December 2008 (FIG. 10B) and the sequence of the ERO1L protein (FIG. 10A) that it encodes (SEQ. ID 6). Related nucleotide sequences are the genomic sequences, AL133453.3 (105038 . . . 158852, complement) and CH471078.2, the mRNA sequences, AF081886.1, AF123887.1, AK292839.1, AY358463.1, BC008674.1, BC012941.1, CR596292.1, CR604913.1, CR614206.1 and CR624423.1 and the protein sequences EAW65646.1, EAW65647.1, AAF35260.1, AAF06104.1, BAF85528.1, AAQ88828.1, AAH08674.1 and AAH12941.1 as deposited or updated on 1 May 2009

FIG. 11: provides the sequence (SEQ. ID 7) of the Homo sapiens fibroblast growth factor 21 (FGF21), mRNA NM_{—}019113 940 bp mRNA with accession NM_{—}019113 (locus NM_{—}019113 940 bp mRNA as deposited on 12 April 2009 (FIG. 11B) and the sequence of the FGF21 fibroblast growth factor 21 protein (FIG. 11A) that it encodes (SEQ. ID 8). Related nucleotide sequences are the genomic sequences, AC009002.5(9604 . . . 11842, complement) and CH471177.1, the mRNA sequences, AB021975.1, AY359086.1 and BC018404.1 and the protein sequences EAW52401.1, EAW52402.1, BAA99415.1, AAQ89444.1 and AAH18404.1 as deposited or updated on 12 April 2009.

FIG. 12: provides the sequence (SEQ. ID 9) of the Homo sapiens methionine adenosyltransferase I, alpha (MAT1A), mRNA with accession NM_{—}000429 (locus NM_{—}000429 3419 bp mRNA as deposited on 29 March 2009 (FIG. 11B) and the sequence of the MAT1A protein (FIG. 12A) that it encodes (SEQ. ID 10). Related nucleotide sequences are the genomic sequences, AL359195.24 and CH471142.2, the mRNA sequences, AK026931.1, AK290820.1, BC018359.1, BM738684.1, BX496326.1, CR600407.1, D49357.1 and X69078.1 and the protein sequences CAI13695.1, CAI13696.1, EAW80396.1, EAW80397.1, BAF83509.1, AAH18359.1, BAA08355.1 and CAA48822.1 as deposited or updated on 27 March 2009

FIG. 13 provides the sequence (SEQ. ID 11) of the Homo sapiens RNA terminal phosphate cyclase-like 1 (RCL1), mRNA with accession NM_{—}005772 (locus NM_{—}005772 2169 bp mRNA as deposited on 11 February 2008 (FIG. 13B) and the sequence of the RNA terminal phosphate cyclase-like 1 protein (FIG. 13A) that it encodes (SEQ. ID 12). Related nucleotide sequences are the genomic sequences, AL158147.17, AL158147.17, AL353151.26 and CH471071.2the mRNA sequences, AF067172.1, AF161456.1, AJ276894.1, AK022904.1, AK225872.1, BC001025.2, CR600925.1, CR612629.1, CR612665.1, CR613074.1, CR623784.1, CR625779.1, DB024289.1, DB448951.1 and EF553527.1 and the protein sequences CAH70317.1, CAH70318.1, CAH70319.1, CAH70320.1, CAH70317.1, CAH70318.1, CAH70319.1, CAH70320.1, CAH72285.1, CAH72286.1, EAW58776.1, EAW58777.1, AAD32456.1, AAF29016.1, CAB89811.1, BAB14300.1, AAH01025.1, and ABQ66271.1 as deposited or updated on 13 March 2009.

FIG. 14 provides the sequence (SEQ. ID 13) of the Homo sapiens WDR45-like (WDR45L), mRNA with accession NM_{—}019613 (locus NM_{—}019613 2596 bp mRNA as deposited on 1 May 2008 (FIG. 14B) and the sequence of the WDR45-like protein (FIG. 14A) that it encodes (SEQ. ID 14). Related nucleotide sequences are the genomic sequences, AC124283.11 (104972 . . . 138797, complement) and CH471099.1 the mRNA sequences, AA861045.1, AF091083.1, AK297477.1, AM182326.1, AY691427.1, BC000974.2, BC007838.1, CN262716.1, CR456770.1, CR593190.1, CR598197.1, CR600994.1 and CR618973.1 and the protein sequences EAW89808.1, EAW89809.1, EAW89810.1, EAW89811.1, EAW89812.1, EAW89813.1, EAW89814.1, AAC72952.1, BAG59898.1, CAJ57996.1, AAV80763.1, CAG33051.1 as deposited or updated on 31 March 2009.

FIG. 15 provides a list of the differentially expressed genes (fold change above 2 and Limma correction p<0.01) in cultures of HepG2 cells exposed to hypoxia (2% O₂) for 72 hours compared to cells grown at 20% O₂. (Array data are deposited at NCBI with accession number GSE15366).

FIG. 16 is a schematic representation of functional interactions obtained for the 7 gene set from STRING 8.0 computer program. The 7 prognostic hypoxia genes (A) and were linked with predicted functional partners (B) and 15 white nodes (C) were included to show the most relevant interactions. (further explanation see text and table 6).

FIG. 17 provides a Kaplan Meier curve: FIG. 17A displays Kaplan-Meier survival curve demonstrating that if a a cut-off value of 0.35 for the hypoxia score (Log Rank test hypoxia score >0.35 (n=42) was 307 days, whereas the median survival for patients with a hypoxia score ≦0.35 (n=93) was 1602 days (p=0.002) and FIG. 17B displays a Kaplan Meier curve showing a significant difference in early recurrence (p=0.005) when the a cut-off of 0.35 for the hypoxia score is used.

### DETAILED DESCRIPTION

### Illustrative Embodiments of the Invention

The present invention provides an in vitro method, for evaluating hypoxia in a HCC tumour and for evaluating a biological stage of an HCC tumour in an individual, based on a sample from the individual, comprising: deriving from the sample a profile data set, the profile data set on the gene expression panel with the marker constituents, CCNG2, EGLN3, ERO1L, FGF21, MAT1A, RCL1 and WDR45L, (i.e. the HCC hypoxia marker genes) or a substantially similar marker for CCNG2, EGLN3, ERO1L, FGF21, MAT1A, RCL1 or WDR45L, being a quantitative measure of the amount of a distinct RNA or protein constituent in the panel so that measurement of the constituents enables evaluation of the biological condition or the biological behaviour of HCC tumours.

As used herein the term “individual” shall mean a human person, an animal or a population or pool of individuals.

As used herein, the term “candidate agent” or “drug candidate” can be natural or synthetic molecules such as proteins or fragments thereof, antibodies, small molecule inhibitors or agonists, nucleic acid molecules e.g. antisense nucleotides, ribozymes, double-stranded RNAs, organic and inorganic compounds and the like.

mRNA expression levels that are expressed in absolute values represent the number of molecules for a given gene calculated according to a standard curve. To perform quantitative measurements serial dilutions of a cDNA (standard) are included in each experiment in order to construct a standard curve necessary for the accurate mRNA quantification. The absolute values (number of molecules) are given after extrapolation from the standard curve.

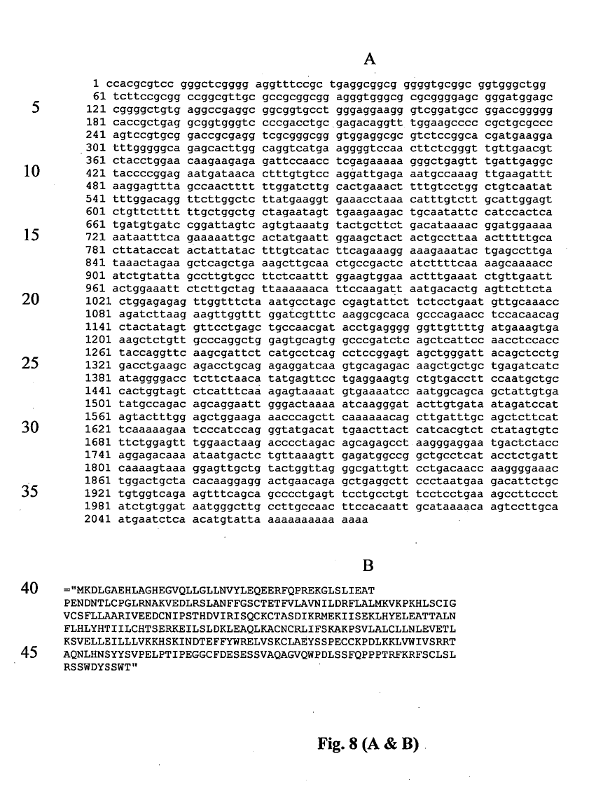

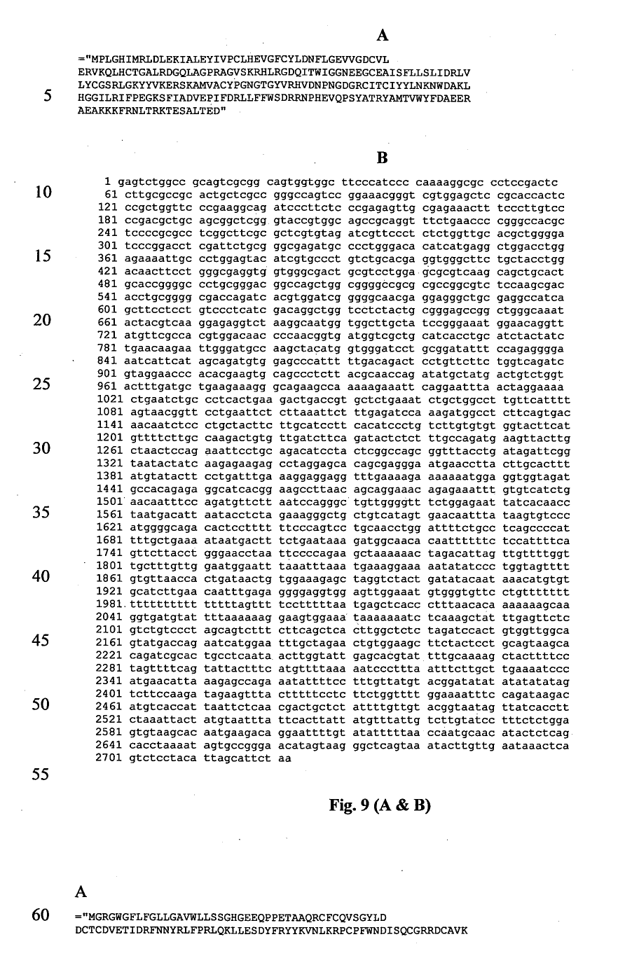

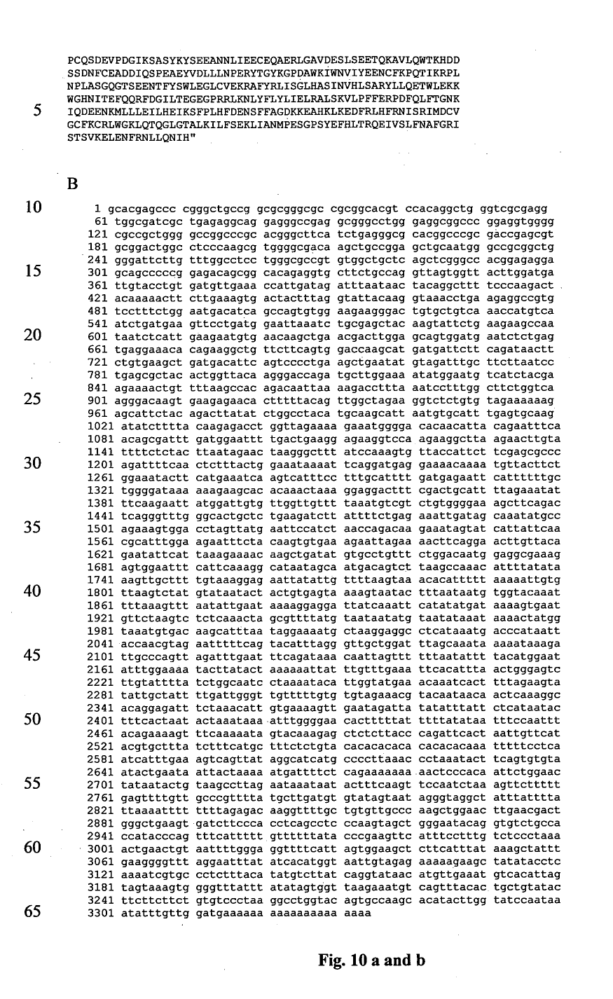

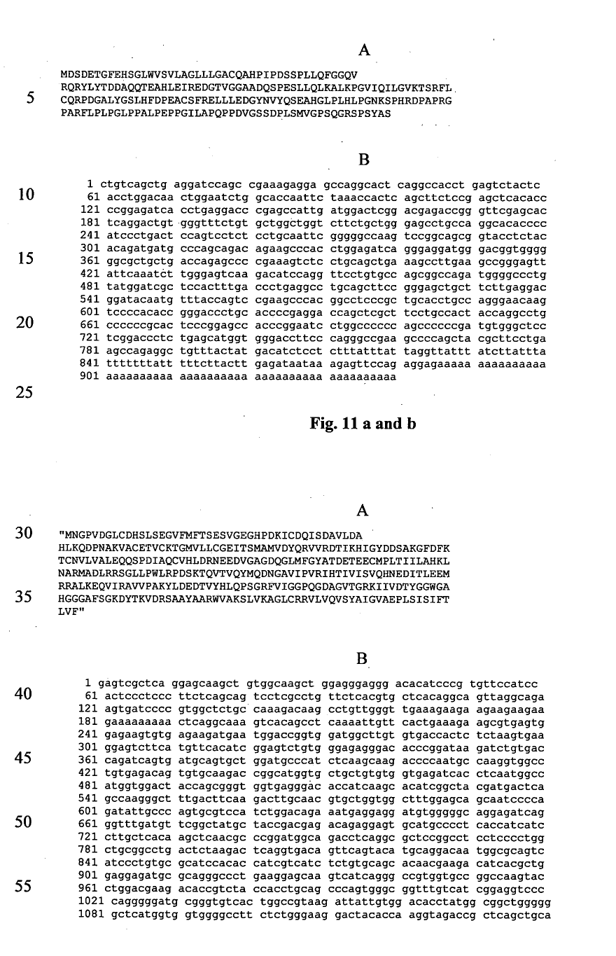

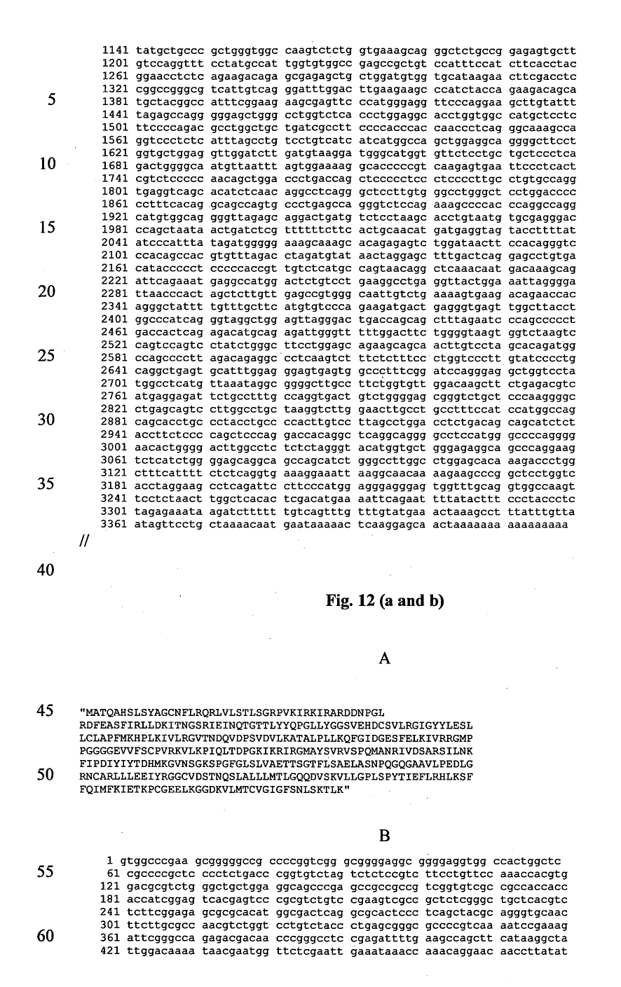

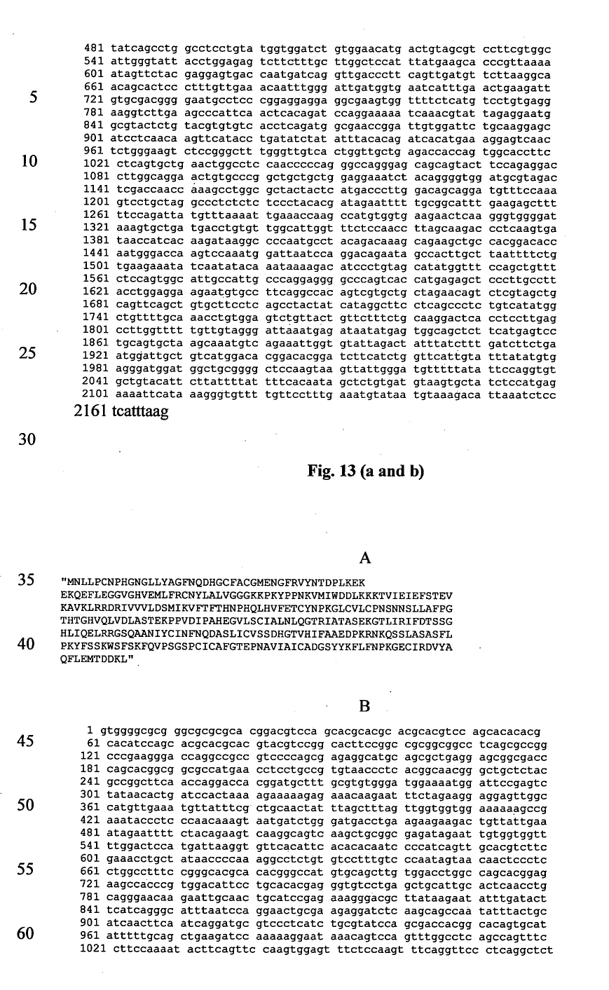

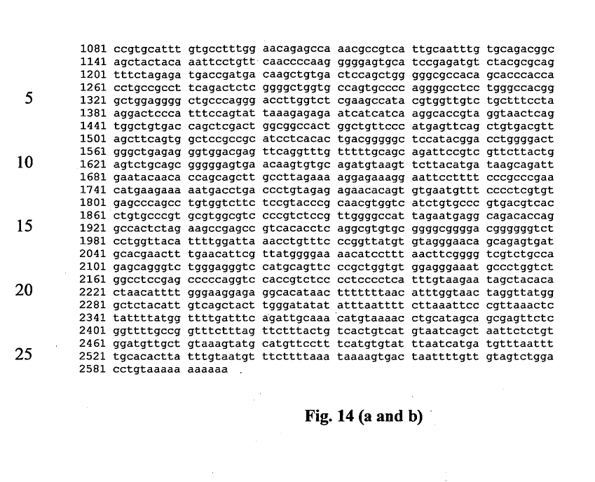

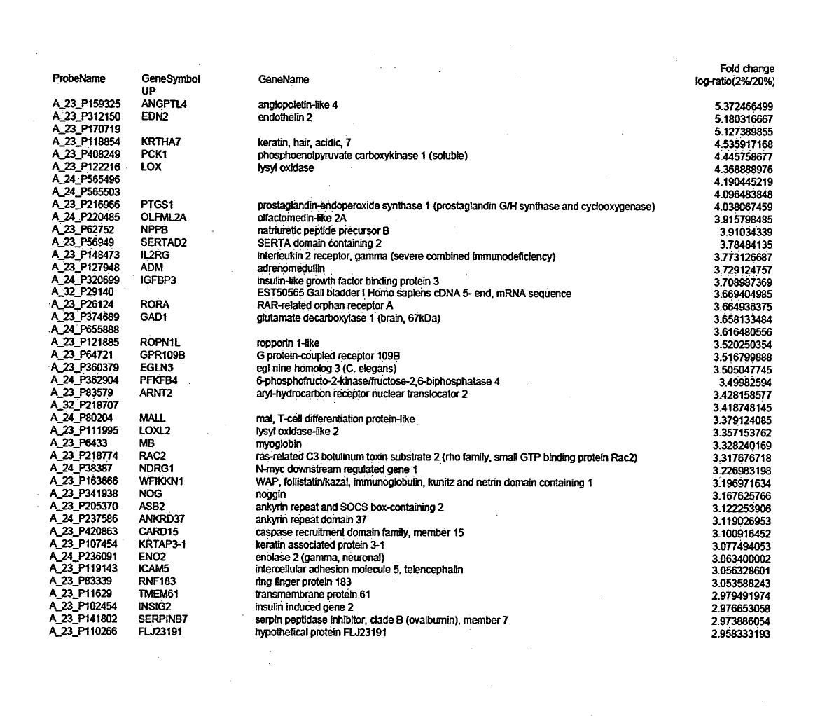

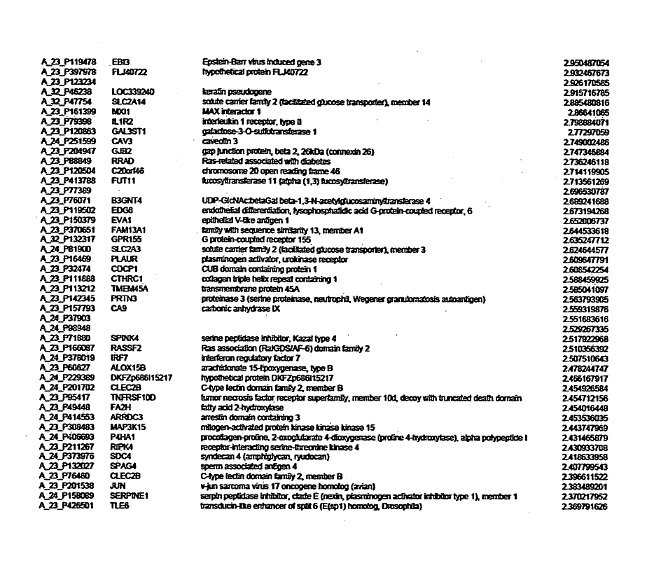

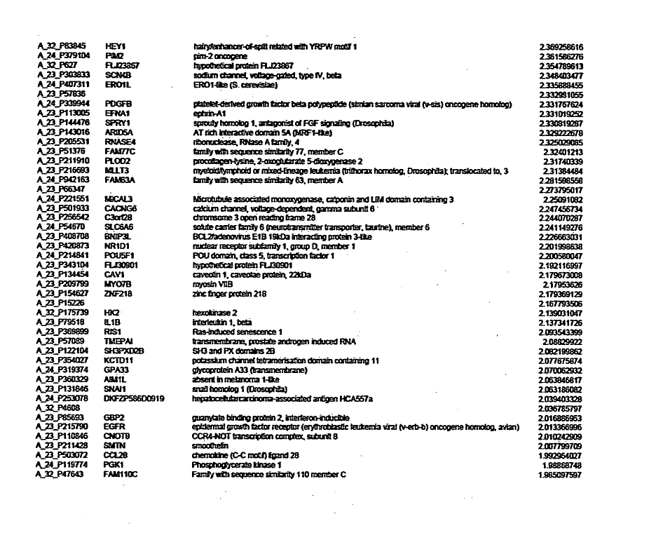

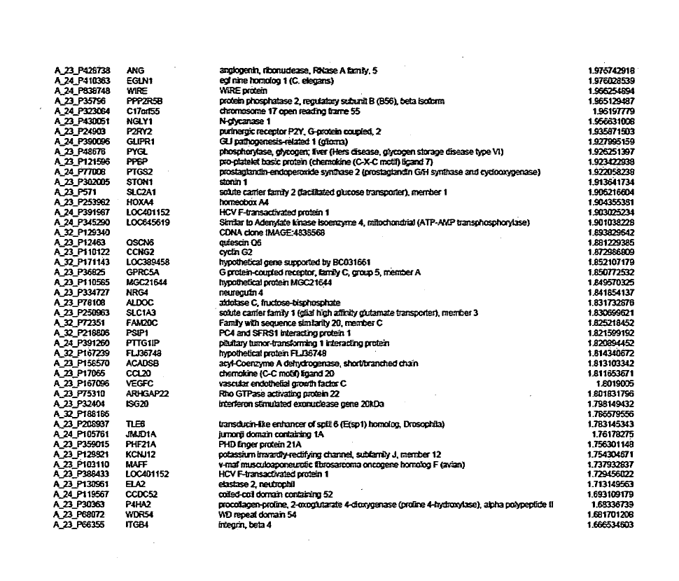

As used herein each marker referred to as CCNG2 (ref. ID's 1 and 2: FIG. 8), EGLN3 (ref. ID's 3 and 4: FIG. 9), ERO1L (ref. ID's 5 and 6: FIG. 10), FGF21 (ref. ID's 7 and 8: FIG. 11), MAT1A (ref. ID's 9 and 10: FIG. 12), RCL1 (ref. ID's 11 and 12: FIG. 13) and WDR45L (ref. ID's 13 and 14: FIG. 14) encompass the gene or gene product (including mRNA and protein) that are substantially similar to these markers

In its broadest sense, the term “substantially similar”, when used herein with respect to a nucleotide sequence, means a nucleotide sequence corresponding to a reference nucleotide sequence, wherein the corresponding sequence encodes a polypeptide having substantially the same structure and function as the polypeptide encoded by the reference nucleotide sequence, e.g. where only changes in amino acids not affecting the polypeptide function occur. Desirably the substantially similar nucleotide sequence encodes the polypeptide encoded by the reference nucleotide sequence. The percentage of identity between the substantially similar nucleotide sequence and the reference nucleotide sequence desirably is at least 80%, more desirably at least 85%, preferably at least 90%, more preferably at least 95%, still more preferably at least 99%. Sequence comparisons are carried out using a Smith Waterman sequence alignment algorithm (see e.g. Waterman, M. S. Introduction to Computational Biology: Maps, sequences and genomes. Chapman & Hall. London: 1995. ISBN 0-412-99391-0).

A nucleotide sequence “substantially similar” to reference nucleotide sequence can also hybridize to the reference nucleotide sequence in 7% sodium dodecyl sulphate (SDS), 0.5 M NaPO4, 1 mM EDTA, pH 7.2 at 50° C. with washing in 2×SSC, 0.1% SDS at 50° C., 20 more desirably in 7% sodium dodecyl sulphate (SDS), 0.5 M NaPO4, 1 mM EDTA, pH 7.2 at 50° C. with washing in 1×SSC, 0.1% SDS at 50° C., more desirably still in 7% sodium dodecyl sulphate (SDS), 0.5 M NaPO4, 1 mM EDTA, pH 7.2 at 50° C. with washing in 0.5×SSC, 0.1% SDS at 50° C., preferably in 7% sodium dodecyl sulphate (SDS), 0.5 M NaPO4, 1 mM EDTA, pH 7.2 at 50° C. with washing in 0.1×SSC, 0.1% SDS at 50° C., more preferably in 7% sodium 25 dodecyl sulphate (SDS), 0.5 M NaPO4, 1 mM EDTA, pH 7.2 at 50° C. with washing in 0.1×SSC, 0.1% SDS at 65° C., yet still encodes a functionally equivalent gene product.

The present invention provides a plurality of markers (CCNG2, EGLN3, ERO1L, FGF21, MAT1A, RCL1 and WDR45L) or substantially similar markers that together, alone or in combinations, are or can be used as markers of the biological behaviour or the stage of a HCC tumour. In a preferred embodiment of the present methods, at least 2 or 3, at least 3 or 4, or at least 5, 6 or 7 markers selected among CCNG2, EGLN3, ERO1L, FGF21, MAT1A, RCL1 and WDR45L can be used for determination of their gene expression profiles. Within the context of the present invention particular subsets of the HCC hypoxia marker genes consist of;
CCNG2 in combination with two, three, four or five marker genes selected of the group consisting of EGLN3, ERO1L, FGF21, MAT1A, RCL1 and WDR45L.
WDR45L in combination with two, three, four or five marker genes marker genes selected of the group consisting of EGLN3, ERO1L, FGF21, MAT1A, RCL1 and CCNG2.
WDR45L in combination with one, two, three, four or five marker genes selected of the group consisting of EGLN3, ERO1L, MAT1A, RCL1 and CCNG2.
MAT1A in combination with one, two, three, four or five marker genes selected of the group consisting of EGLN3, ERO1L, FGF21, WDR45L, RCL1 and CCNG2.
RCL1 optionally in combination with one, two, three, four or five marker genes selected of the group consisting of EGLN3, ERO1L, FGF21, MAT1A, WDR45L and CCNG2.
RCL 1 in combination with one, two, three, four or five marker genes selected of the group consisting of EGLN3, ERO1L, MAT1A, WDR45L and CCNG2.
RCL1 in combination with MAT1A.
RCL1 in combination with WDR45L
RCL1 in combination with MAT1A, and WDR45L.
The combination of the seven marker genes consisting of CCNG2, EGLN3, ERO1L, FGF21, MAT1A, RCL1 and WDR45L

In particularly useful embodiments, a plurality of these markers can be selected and their mRNA expression monitored simultaneously to provide expression profiles for use in various aspects.

In a further preferred embodiment of the present methods, mRNA expression is assessed in the HCC tumour tissues by techniques selected from the group consisting of Northern blot analysis, reverse transcription PCR, real time quantitative PCR, NASBA, TMA, medium-high throughput gene expression quantification system for instance using microarrays and real-time reverse transcriptase (RT)-PCR, digital mRNA profiling (Fortina 2008) or any other available amplification technology. In each of said methods, the means to determine the level of mRNA expression include one or more oligonucleotides specific for the HCC hypoxia marker genes. In contrast to the hybridization conditions to determine the sequene similarity of “substantially similar” nucleotide sequences, these techniques are usually performed with relatively short probes (e.g., usually about 16 nucleotides or longer for PCR or sequencing and about 40 nucleotides or longer for in situ hybridization). The high stringency conditions used in these techniques are well known to those skilled in the art of molecular biology, and examples of them can be found, for example, in Ausubel et al., Current Protocols in Molecular Biology, John Wiley & Sons, New York, N.Y., 1998, which is hereby incorporated by reference.

A “probe” or “primer” is a single-stranded DNA or RNA molecule of defined sequence that can base pair to a second DNA or RNA molecule that contains a complementary sequence (the target). The stability of the resulting hybrid molecule depends upon the extent of the base pairing that occurs, and is affected by parameters such as the degree of complementarity between the probe and target molecule, and the degree of stringency of the hybridization conditions. The degree of hybridization stringency is affected by parameters such as the temperature, salt concentration, and concentration of organic molecules, such as formamide, and is determined by methods that are known to those skilled in the art. Probes or primers specific for the nucleic acid biomarkers described herein, or portions thereof, may vary in length by any integer from at least 8 nucleotides to over 500 nucleotides, including any value in between, depending on the purpose for which, and conditions under which, the probe or primer is used. For example, a probe or primer may be 8, 10, 15, 20, or 25 nucleotides in length, or may be at least 30, 40, 50, or 60 nucleotides in length, or may be over 100, 200, 500, or 1000 nucleotides in length. Probes or primers specific for the nucleic acid biomarkers described herein may have greater than 20-30% sequence identity, or at least 55-75% sequence identity, or at least 75-85% sequence identity, or at least 85-99% sequence identity, or 100% sequence identity to the nucleic acid biomarkers described herein. Probes or primers may be derived from genomic DNA or cDNA, for example, by amplification, or from cloned DNA segments, and may contain either genomic DNA or cDNA sequences representing all or a portion of a single gene from a single individual. A probe may have a unique sequence (e.g., 100% identity to a nucleic acid biomarker) and/or have a known sequence. Probes or primers may be chemically synthesized. A probe or primer may hybridize to a nucleic acid biomarker under high stringency conditions as described herein.

Probes or primers can be detectably-labeled, either radioactively or non-radioactively, by methods that are known to those skilled in the art. Probes or primers can be used for lung cancer detection methods involving nucleic acid hybridization, such as nucleic acid sequencing, nucleic acid amplification by the polymerase chain reaction (e.g., RT-PCR), single stranded conformational polymorphism (SSCP) analysis, restriction fragment polymorphism (RFLP) analysis, Southern hybridization, northern hybridization, in situ hybridization, electrophoretic mobility shift assay (EMSA), fluorescent in situ hybridization (FISH), and other methods that are known to those skilled in the art.

By “detectably labelled” is meant any means for marking and identifying the presence of a molecule, e.g., an oligonucleotide probe or primer, a gene or fragment thereof, or a cDNA molecule. Methods for detectably-labelling a molecule are well known in the art and include, without limitation, radioactive labelling (e.g., with an isotope such as 32P or 35S) and nonradioactive labelling such as, enzymatic labelling (for example, using horseradish peroxidase or alkaline phosphatase), chemiluminescent labeling, fluorescent labeling (for example, using fluorescein), bioluminescent labeling, or antibody detection of a ligand attached to the probe. Also included in this definition is a molecule that is detectably labeled by an indirect means, for example, a molecule that is bound with a first moiety (such as biotin) that is, in turn, bound to a second moiety that may be observed or assayed (such as fluorescein-labeled streptavidin). Labels also include digoxigenin, luciferases, and aequorin.

In another preferred embodiment of the present methods, the level of gene expression can alternatively be assessed by detecting the presence of a protein corresponding to the gene expression product, and typically includes the use of one or more antibodies specific for a protein encoded by the HCC hypoxia marker genes.

An antibody “specifically binds” an antigen when it recognizes and binds the antigen, for example, a biomarker as described herein, but does not substantially recognize and bind other molecules in a sample. Such an antibody has, for example, an affinity for the antigen, which is at least 2, 5, 10, 100, 1000 or 10000 times greater than the affinity of the antibody for another reference molecule in a sample. Specific binding to an antibody under such conditions may require an antibody that is selected for its specificity for a particular biomarker. For example, a polyclonal antibody raised to a biomarker from a specific species such as rat, mouse, or human may be selected for only those polyclonal antibodies that are specifically immunoreactive with the biomarker and not with other proteins, except for polymorphic variants and alleles of the biomarker. In some embodiments, a polyclonal antibody raised to a biomarker from a specific species such as rat, mouse, or human may be selected for only those polyclonal antibodies that are specifically immunoreactive with the biomarker from that species and not with other proteins, including polymorphic variants and alleles of the biomarker. Antibodies that specifically bind any of the biomarkers described herein may be employed in an immunoassay by contacting a sample with the antibody and detecting the presence of a complex of the antibody bound to the biomarker in the sample. The antibodies used in an immunoassay may be produced as described herein or known in the art, or may be commercially available from suppliers, such as Dako Canada, Inc., Mississauga, ON. The antibody may be fixed to a solid substrate (e.g., nylon, glass, ceramic, plastic, etc.) before being contacted with the sample, to facilitate subsequent assay procedures. The antibody-biomarker complex may be visualized or detected using a variety of standard procedures, such as detection of radioactivity, fluorescence, luminescence, chemiluminescence, absorbance, or by microscopy, imaging, etc. Immunoassays include immunohistochemistry, enzyme-linked immunosorbent assay (ELISA), western blotting, immunoradiometric assay (IRMA), lateral flow, evanescence (DiaMed AG, Cressier sur Morat, Switzerland, as described in European Patent Publications EP1371967, EP1079226 and EP1204856), immuno histo/cyto-chemistry and other methods known to those of skill in the art. Immunoassays can be used to determine presence or absence of a biomarker in a sample as well as the amount of a biomarker in a sample. The amount of an antibody-biomarker complex can be determined by comparison to a reference or standard, such as a polypeptide known to be present in the sample. The amount of an antibody-biomarker complex can also be determined by comparison to a reference or standard, such as the amount of the biomarker in a reference or control sample. Accordingly, the amount of a biomarker in a sample need not be quantified in absolute terms, but may be measured in relative terms with respect to a reference or control.

While individual HCC hypoxia markers, such as in particular RCL1, are useful in determining Hypoxia in an HCC tumour, the combination of HCC hypoxia biomarkers as proposed herein enables accurate determination of the hypoxic response of an HCC tumour. The profile data set(s) as proposed herein, achieves such measure for each constituent under measurement conditions that are substantially repeatable and wherein specificity and efficiencies of amplification for all constituents are substantially similar. As is known to the person skilled in the art any suitable statistical methods and algorithms, e.g., logistical regression algorithm (Applied Logistic Regression, David W. Hosmer & Stanley Lemesho, Wiley-Interscience, 2nd edition, 2001 and Applied multivariate techniques, Subhash Sharma, John Wiley & Sons, Inc, 1996), may be used to analyse and use the profile data set of the CCNG2, EGLN3, ERO1L, FGF21, MAT1A, RCL1 and WDR45L markers, for providing an index that is indicative of the biological condition, i.e. the hypoxic response of the HCC tumour, or of the biological behaviour of the HCC tumour, i.e. the invasiviness/morbidity of the HCC tumour in said individual. In each of the aforementioned methods, the expression profiles will be compared to a control, such as a set of predetermined standard values of the expression of said genes in a normal cell e.g., a cell derived from a subject without cancer or with undetectable cancer or a normal cell derived from a subject who has undergone successful resection of HCC. Alternatively the in vitro method provides with the index a normative value of the index function, determined with respect to a relevant population of HCC samples, so that the index may be interpreted in relation to the normative value for a biological condition of HCC.

Another aspect of the invention is a kit for use in a diagnosis of the biological behaviour of a HCC tumour in an individual. Such kit for use in a diagnosis of the biological behaviour of a HCC tumour in an individual can comprise a means for determining the level of gene expression corresponding to CCNG2 and determining the level of gene expression corresponding to at least two, three, four or five marker genes selected of the group consisting of EGLN3, ERO1L, FGF21, MAT1A, RCL1 and WDR45L.

The kit for use in a diagnosis of the biological behaviour of a HCC tumour in an individual may alternatively comprise a means for determining the level of gene expression corresponding to WDR45L and determining the level of gene expression corresponding to at least two, three, four or five marker genes marker genes selected of the group consisting of EGLN3, ERO1L, FGF21, MAT1A, RCL1 and CCNG2.

Yet another embodiment of present invention is kit for use in a diagnosis of the biological behaviour of a HCC tumour in an individual that comprises a means for determining the level of gene expression corresponding to RCL1 and determining the level of gene expression corresponding to at least one, two, three, four or five marker genes marker genes selected of the group consisting of EGLN3, ERO1L, FGF21, MAT1A, WDR45L and CCNG2.

The most preferred kit of the present invention concerns a kit for use in a diagnosis of the biological behaviour of a HCC tumour in an individual that comprises a means for determining the level of gene expression corresponding to the marker genes selected of the group consisting of CCNG2, EGLN3, ERO1L, FGF21, MAT1A, RCL1 and WDR45L.

The above-described kits can comprise of one or more oligonucleotides specific for a marker gene of the group consisting of CCNG2, EGLN3, ERO1L, FGF21, MAT1A, RCL1 and WDR45L for the determination of the level of gene expression of the selected marker gene. Alternatively, the above-described kits comprise one or more antibodies specific for a protein encoded by a marker gene of the group consisting of CCNG2, EGLN3, ERO1L, FGF21, MAT1A, RCL1 and WDR45L for the determination of the level of gene expression of the selected marker gene.

In such kit the antibody can be selected among polyclonal antibodies, monoclonal antibodies, humanized or chimeric antibodies, and biologically functional antibody fragments (such as single chain, Fab, fab2 or nanobodies™) sufficient for binding of the antibody fragment to the EGLN3, ERO1L, RCL1, FGF21, MAT1A, WDR45L and CCNG2 markers or substantially similar markers. In a particular embodiment of present invention the kit for determining the level of gene expression comprise an immunoassay method. Eventually such kit comprises a means for obtaining a HCC tumour sample of the individual. The above-described kits can further comprise a container suitable for containing the means for determining the level of gene expression and the body sample of the individual. Eventually such kits comprise an instruction for use and interpretation of the kit results.

Still another aspect of the invention is a method for determining the biological behaviour of a HCC tumour from an individual comprising: (a) obtaining a test HCC tumour sample from said individual, (b) determining from the test sample the level of gene expression corresponding to all 7 genes selected among CCNG2, EGLN3, ERO1L, FGF21, MAT1A, RCL1 and WDR45L or more genes; or any of the subsets/combinations of said genes according to the present invention, to obtain a first set of value, and (c) comparing the first set of value with a second set of value corresponding to the level of gene expression assessed for the same gene(s) and under identical condition as for step b) in a HCC tumour sample with a defined biological behaviour history to define the biological behaviour of said test HCC tumour and/or to define a suitable candidate agent or drug candidate to treat said HCC.

Molecular biology techniques and tools used in the aforementioned genetic diagnoses including enzymatic tools for in vitro treatment of DNA; DNA fragmentation; Separation of DNA fragments by electrophoresis and membrane transfer; Selective amplification of a nucleotide sequence; DNA sequence amplification by PCR; RNA amplification as cDNA by RT-PCR; Quantitative PCR methods; RNA or DNA isothermic NASBA R amplification; DNA fragment ligation: recombinant DNA and cloning; DNA cloning, the cloning vectors; DNA fragment sequencing; reading of the sequencing reaction products; molecular hybridization techniques and applications; probes, labelling and reading of the signal; FISH and in situ PCR; detection and dosage methods using signal amplification; southern blot hybridization; ASO techniques: dot blot and reverse-dot blot; ARMS and OLA techniques; DNA microarrays; denaturing gradient gel electrophoresis (DGGE); genetic tests for cancer predisposition; polymerase chain reactions; real-time polymerase chain reaction and melting curve analysis; in-cell polymerase chain reaction; qualitative and quantitative DNA and RNA analysis by matrix-assisted laser desorption/ionization time-of-flight mass spectrometry; polymerase chain reaction products by denaturing high-performance liquid chromatography etc. . . . are available to the man skilled in the arts in manuals such as Diagnostic Techniques in Genetics Edited by Jean-Louis Serre 2006 John Wiley & Sons Ltd; Clinical Applications of PCR Second Edition Edited by Y. M. Dennis Lo, Rossa W. K. Chiu and K. C. Allen Chan 2006 Humana Press Inc.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### EXAMPLES

### Example 1

### Examples Summarized

Methods—Human hepatoblastoma cells HepG2 were cultured in either normoxic (20% O₂) or hypoxic (2% O₂) conditions for 72 hrs, the time it takes to adapt to chronic hypoxia. After 3 days the cells were harvested and analyzed by microarray technology. The highly significant differentially expressed genes were selected and used to assess the clinical value of our in vitro chronic hypoxia gene signature in four published patient studies. Three of these independent microarray studies on HCC patients were used as training sets to determine a minimal prognostic gene set and one study was used for validation. Gene expression analysis and correlation with clinical outcome was assessed with the bioinformatics method of Goeman et al (Goeman 2004).

Results—In the HepG2 cells, 2959 genes were differentially expressed in cells cultured at 2% oxygen for 72 hrs. Out of these, 265 showed a high significant change (2-fold change and Limma corrected p≦0.01). The level of gene expression after 72 hrs was different from the acute hypoxic response (during the first 24 hours) and represented chronicity. Using computational methods we identified 7 out of the 265 highly significant genes that showed correlation with prognosis in all three different training sets and this was independently validated in a 4th dataset. With our approach we could include the largest number of HCC patients in one single study.

Conclusion—We identified a 7-gene signature, which is associated with chronic hypoxia and predicts prognosis in patients with HCC for diagnosing and predicting the biological behaviour of HCC, to determine based on the biological behaviour of the HCC tumour the most suitable therapy and for guiding the development in new HCC therapeutics.

### Example 2

### Molecular Classification

Several studies have tried to identify gene sets with prognostic or diagnostic relevance by microarray analysis. Each study resulted in its own classification with a specific separation into clusters. Some general mechanisms came forward in most of these studies: the proliferation cluster with upregulation of the mTOR pathway, and the beta-catenin cluster. Classification of HCC was not merely done on primary tumours, but it has also been performed on surrounding tissue to determine the risk of recurrence after surgical resection of the primary lesion (Hoshida 2008, Budhu 2006). In the surrounding tissue it appears that genes involved in the inflammatory response predict recurrence. Nevertheless, it is difficult to cluster all the HCCs into these recently identified subgroups and to find a clear correlation between the molecular class and prognosis. All these microarray studies show remarkable little overlap. The first major obstacle is the limited number of patients and different etiologies from which both clinical and corresponding molecular data are available. The results of the studies seem to be centre dependent for several reasons. First of all different microarray techniques are used. Secondly, small heterogeneous cohorts are studied and thirdly, different clinical parameters are used for the evaluation (Ein-Dor 2006). Using modern data analysis techniques, we could evaluate the data from all the major array studies to date on HCC and studied the role of chronic hypoxia as a common mechanism regulating gene expression and determining prognosis.

### Example 3

### Microenvironment and Hypoxia

The microenvironment plays a role in tumour biology but has not been studied extensively in HCC. One of the microenvironmental factors that appear to affect cancer cell behaviour and patient prognosis is hypoxia (Gort 2008). Although HCC is a hypervascular malignancy, there are regions with hypoxia as also seen in other solid tumours (Brown 1998). Hypoxic regions are already present in the early stage when the vasculature is not sufficient extended and in more advanced stages when the rapid cell proliferation induces hypoxia (Kim 2002). Moreover, liver cancer develops usually in a cirrhotic environment where the blood flow is already impaired and more importantly, during the expansion of the tumour the neovascularisation is unorganized with leaky blood vessels, arteriovenous shunting, large diffusion distances and coiled vessels. These structural and functional defects lead to both acute hypoxia due to fluctuating flow and to chronic hypoxia due to diffusion distances of more than 150 μm (Brahimi-Horn 2007, Folkman 2000, Brown 1998).

Hypoxia is associated with poor prognosis in several malignancies, such as cervix and breast carcinoma and with the development of resistance to chemotherapeutic agents and radiation (Semenza 2003, Brown 2004). Hypoxia induces a transcription response that is mainly initiated by hypoxia inducible factor-1 alpha (HIF1A). In normoxic conditions HIF1A is rapidly broken down in the cytoplasm through ubiquitination by the cooperation between Von Hippel Lindau protein and the oxygen sensors prolylhydroxylase (PHD) and factor inhibiting HIF (FIH). When oxygen is lacking, HIF1A accumulates and can translocate to the nucleus and form the transcriptionally active complex HIF1 by coupling to HIF1B (also ARNT). HIF1 is a master control gene with over fifty target genes and alters different pathways (example of a gene involved is between brackets), such as angiogenesis (VEGF), glycolysis (GLUT1), apoptosis (BNIP) and cell proliferation (IGF2) among others (Semenza 2003). Hitherto, studies evaluated only the early changes in gene expression of cells exposed to maximum 24 hours of hypoxia (Fink 2001, Vengellur 2005, Sonna 2003). We hypothesized that during the development of HCC there are regions with sustained hypoxia and that these tumours have a gene expression pattern corresponding with chronic reduced oxygen. And further, that the grade of hypoxic gene expression determines the grade of aggressiveness, or more in general, the prognosis. Our aim was to develop a widely applicable gene set that represents chronic hypoxia and that has prognostic relevance. So, we developed an experimental model for chronic hypoxia in the HepG2 liver cell line. In this model we show by real-time PCR and immunohistochemistry that the in vitro signature for a set of hypoxia related genes under chronic hypoxia differs from acute hypoxia. We characterized the long-term (72 hrs) changes in gene expression in HepG2 cells by microarray analysis. Using computational data analysis techniques such as the global test as described by Goeman et al (Goeman 2004) we could evaluate the data from all the major array studies to date on HCC.

We were able to study the role of chronic hypoxia as a common mechanism regulating gene expression and determining prognosis in a very robust manner.

### Example 4

### Materials and methods

Cell Culture

HepG2 human hepatoblastoma cells were obtained from ATCC (HB-8065, Rockville, Md., USA). Cells were grown in a humidified incubator (20% O₂, 5% CO₂ at 37° C.) in Williams Medium E (WEM, InVitrogen) supplemented with 10% foetal calf serum, 2 mM L-glutamine, 20 mU/ml insulin, 50 nM dexamethasone, 100 U/ml penicillin, 100 μg/ml streptomycin, 2.5 μg fungizone, 50 μg/ml gentamycin and 100 μg/ml vancomycin (=WEM−C).

For the microarray analysis two experiments were executed in parallel. Cells were seeded at 3×10⁶ in 75 cm² tissue culture flasks (n=4) at 20% O₂ and were grown until 70% confluence (during five days, with medium refreshment every two days). After reaching near-confluence, cells were washed with buffer and medium was refreshed, 2 flasks were placed in a humidified incubator with hypoxic conditions (2% O₂, 5% CO₂ at 37° C.), while the other flasks (n=2) remained in normoxic conditions (20% O₂). Cells were cultured for 72hrs in these different oxygen conditions and after three days cells were harvested after trypsin treatment, mixed with Trizol (InVitrogen, Merelbeke, Belgium) and stored in −80° C. for further analysis.

Sample Collection and Microarray Target Synthesis and Processing Samples in Trizol were homogenized in a Dounce homogenizer for RNA extraction. Thereafter, RNA was isolated with the RNeasy Kit (Qiagen, Chatsworth, Calif.) according to the manufacturer's instructions. The quality of all RNA samples was monitored by measuring the 260/280 and 260/230 nm ratios with a NanoDrop spectrophotometer (NanoDrop Technologies, Centreville, Del.) and by means of the Agilent 2100 BioAnalyzer (Agilent, Palo Alto, Calif.). Only RNA showing no signs of degradation or impurities (260/280 and 260/230 nm ratios, >1.8) was considered suitable for microarray analysis and used for labelling. Briefly, from 1 μg of cellular RNA, poly-A RNA was reversed transcribed using a poly dT-T7 primer. The resulting cDNA was immediately used for one round of amplification by T7 in vitro transcription reaction in the presence of Cyanine 3-CTP or Cyanine 5-CTP. The amplified and labelled RNA probes were purified separately with RNeasy purification columns (Qiagen, Belgium). Probes were verified for amplification yield and incorporation efficiency by measuring the RNA concentration at 280 nm, Cy3 incorporation at 550 nm and Cy5 incorporation at 650 nm using a Nanodrop spectrophotometer.

Samples were hybridized on dual colour Agilent's Human Whole Genome Oligo Microarray (Cat# G4112F, Agilent, Diegem, Belgium) that contained 44 k 60-mer oligonucleotide probes representing around 41,000 well-characterized human transcripts. Agilent technology utilizes one glass array for the simultaneous hybridization of two populations of labelled, antisense cRNAs obtained from two samples (reference and assay).

Primary Data Analysis

Statistical data analysis was performed on the processed Cy3 and Cy5 intensities, as provided by the Feature Extraction Software version 9.1. Probes with none of the eight signals flagged as positive and significant (by the Feature Extraction Software) were omitted from all subsequent analyses as well as the various controls. Further analysis was performed in the R programming environment, in conjunction with the packages developed within the Bioconductor project (http://www.bioconductor.org; Gentleman 2004). In a first analysis the differential expression of the 2% versus 20% oxygen samples was assessed via the moderated t-statistic, described in Smyth (2004). This moderated statistic applies an empirical Bayesian strategy to compute the gene-wise residual standard deviations and thereby increases the power of the test, especially beneficial for smaller data sets. To control the false discovery rate, multiple testing correction was performed and probes with a corrected p-value below 0.05 and a fold change of >2 were selected (Benjamini & Hochberg, 1995). To determine the highly significant differentially expressed genes under chronic hypoxic conditions we used higher stringency with a cut-off fold change of >2 and Limma correction for multiple testing p <0.01. Since multiple probes can correspond to the same gene, the mean value for each gene was calculated after this correction. Finally, the remaining differentially expressed genes were designated as the liver hypoxia gene set and with these genes we could further investigate the relevance of chronic hypoxia in primary human liver cancer.

Cell Metabolism

Cell metabolism under different oxygen concentrations was assessed comparing cell number (determined by Coulter counter, Beckman, Fullerton Calif., USA)) and metabolic activity (determined by XTT-assay, Roche, Vilvoorde Belgium). First the metabolic response to acute hypoxia was determined. HepG2 cells were cultured at 20% O₂, harvested by trypsin treatment and cell number was determined. Cells were seeded in two 24 well plates in different cell numbers and incubated with XTT-solution for 4 hours at either normoxic or hypoxic conditions, hereafter medium was harvested, spinned off and placed in a 96-well plate to determine metabolism in the plate reader (490 nm/ref 655 nm Biorad Model 3550, Hercules, Calif., USA).

For the metabolic activity after chronic hypoxia (72 hours at 2% O₂) HepG2 cells were grown in 75 cm² tissue culture flasks and at near confluence placed in either normoxic (control) or hypoxic conditions. After 72 hrs cells were trypsinized, counted and seeded in a 24 well-plate in different cell numbers. Cells were incubated with XTT-solution for additional 4 hours, still in their original oxygen condition. After 4 hrs medium was harvested, and transferred into a 96 well plate in triplicate to determine metabolic activity in the plate reader.

Quantitative RT-PCR

To investigate the dynamics of hypoxia related gene expression and to confirm the array findings, we performed RT-PCR at different time points for several selected genes (n=10 or table 1). HepG2 cells were seeded in 25 cm³ culture flasks (10⁶ cells/flask), using the same culture conditions as were used for the microarray experiment. The experiment started when cells had reached 70% confluency. Medium was refreshed and flasks were placed in either 2% O₂ or 20% O₂. Gene expression was tested at 0 hr, 10 hrs, 24 hrs and up to 72 hrs. All culture conditions were performed in triplicate and cells were collected for RNA isolation.

Two genes that were top listed as upregulated gene and three genes that were top listed as downregulated were selected. Furthermore, we tested different well-known hypoxia inducible genes and beta-2-microglobulin was used as housekeeping gene. RNA was isolated with the RNeasy Kit (Qiagen, Chatsworth, Calif.) according to the manufacturer's instructions. One microgram of cellular RNA was reverse transcribed into cDNA using SuperScript II reverse transcriptase and random hexamer primers (Invitrogen Life Technologies, USA).

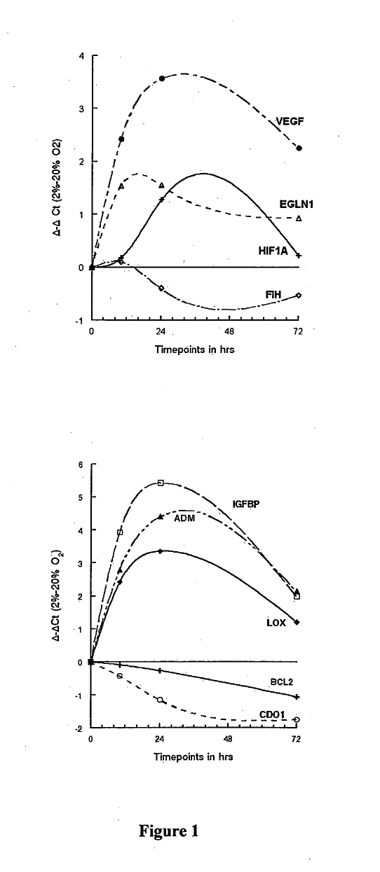

The PCR reaction was carried out in a volume of 25 μl in a mixture that contained appropriate sense- and anti-sense primers and a probe in TaqMan Universal PCR Master Mixture (Applied Biosystems, Foster City, Calif.). We used the Assays-on-Demand™ Gene Expression products, which consist of a 20×mix of unlabeled PCR primers and TaqMan MGB probe (FAM™ dye-labelled). These assays are designed for the detection and quantification of specific human genetic sequences in RNA samples converted to cDNA (The primer references (Applied Bioscience) are listed in table 1). Real-time PCR amplification and data analysis were performed using the A7500 Fast Real-Time PCR System (Applied Biosystems). Each sample was assayed in duplicate in a MicroAmp optical 96-well plate. The thermo-cycling condition consisted of 2 minutes at 50° C. and 10 min incubations at 95° C., followed by 40 two-temperature cycles of 15 seconds at 95° C. and 1 min at 60° C. The ΔΔCt-method was used to determine relative gene expression levels (FIGS. 1A and 1B).

Immunohistochemistry on HIF1A and VEGF

HepG2 cells were grown on Thermanox plastic cover slips (Nalgene Nunc international, Rochester, N.Y. USA, 13 mm diameter) placed in a 24 well plate with 1 mL William's Medium E (WEM-C, InVitrogen). After one day of incubation and attachment, cells were either exposed to hypoxia (2% O₂) or normal oxygen conditions for 0, 24, or 72 hours. Subsequently cells were washed once with PBS and fixed in acetone for 15 minutes. When dry, the cover slides were stored at −20° C.

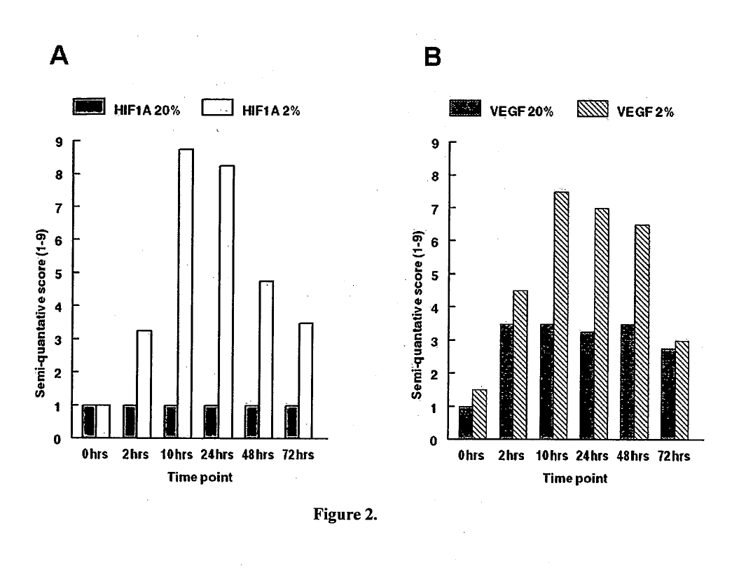

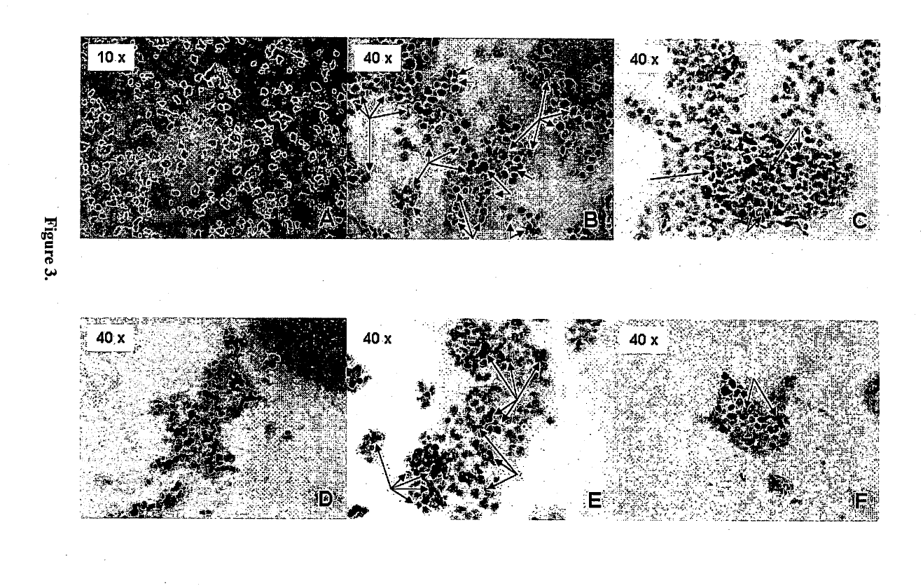

For immunohistochemistry we used the Envision technique of Dako. Cover slips collected at the different time points were stained in duplicate. Cells were incubated for 45 minutes with a primary antibody against HIF1 A (1:250 anti-HIF1 Amonoclonal mouse antibody, BD Biosciences) or against VEGF (1:100 anti-VEGF A-20 polyclonal rabbit antibody, Santa Cruz). As secondary antibody Envision monoclonal antibodies were used (for HIF1A; Envision monoclonal mouse antibody, Dako and for VEGF; Envision monoclonal rabbit antibody, Dako). Finally, the staining was performed with 3-amino-9-ethylcarbazole (AEC) for HIF1A and with 3,3′-Diaminobenzidine (DAB) for VEGF and the contra-staining with haematoxylin. The thermanox cover slips were mounted with glycergel. To evaluate the staining we used a semi-quantitative quickscore (Detre 1995) which combines positivity (P) and intensity (I). Positivity was scored as: 1=0-4%, 2=5-19%, 3=20-39%, 4=40-59%, 5=60-79% and 6=80-100%. Intensity was scored as: 0=negative, 1=weak, 2=intermediate and 3=strong. The final score was the total of P+I and has a range of 1-9. All slides were scored independently by two researchers (FIGS. 2A and 2B).

Gene Expression in HCC Patient Studies

The heterogeneous nature of HCC, the analytical aspects of the different DNA microarray technologies together with the use of different clinical criteria have made it difficult to accurately and reproducibly classify HCC (Thorgeirsson 2006). Furthermore, most studies use a “top-down” approach, where small patient groups are hierarchical clustered based on thousands of genes. The predictive gene lists that are extracted with this method highly depend on patient selection (Chang 2005, Liu 2005). To overcome these disadvantages we aimed to develop an array-platform independent method of analysis using objective and robust criteria, based on the hypothesis that hypoxia is a general mechanism during HCC expansion. This mechanism-driven method is a “bottom-up” approach to define a prognostic gene list. In order to determine the clinical relevance of the in vitro gene expression we compared our findings with all microarray data sets with corresponding clinical information that are available in public databases.

Until now there are four important publicly available datasets for HCC patients, published in Gene Expression Omnibus (GEO) (Edgar 2002) and Array Express (Parkinson 2008). All these studies used different methods to assess gene expression. The datasets are independent of each other and harbour different clinical and pathological information, such as underlying pathology, tumour size, vascular invasion and FAL-index (table 2).

Two groups used only hepatitis C patients (Wurmbach 2007, Chiang 2008), while the other two included patients with HCC based on different etiologies. The aims of the studies were also different. Lee et al. (Lee 2004, Lee 2006) conducted an analysis on the prognostic value of microarray, Boyault et al. (Boyault 2007) focused on the altered pathways and divided patients into different subgroups, Wurmbach et al. analyzed the different stages of HCC development and included dysplastic and cirrhotic liver tissue as well, whereas Chiang et al. focused on the gene expression profiles of early HCV-induced HCC.

We used the first three published datasets as training sets to optimize our in vitro hypoxia gene set (265 genes) and to investigate the prognostic correlation. The last dataset, Chiang, was used to independently validate the signature. To define a robust score from these different datasets, we used a global test (Goeman, 2004) to investigate whether the hypoxia genes are associated with the prognosis under a Q2 null hypothesis (Tian, 2005). This approach should give the advantage to be less dependent on the array platform used in different laboratories (Affymetrix, Agilent, Stanford etc). Moreover, by starting from a small subset of in vitro determined hypoxia genes, this method provides more insight in the degree of relationship between the different genes found to be up- or downregulated. This method was then used to investigate whether the genes in our hypoxia set separate the good and poor prognostic characteristics in the three datasets individually. So far, no gold standard has been available to predict prognosis, but several factors have been proven to significantly influence outcome. Since in all four datasets another prognostic factor was reported, we also had to use a different prognostic factor in every dataset. From Boyault et al. the FAL-index (Dvorchik 2008, Wilkens 2004) was used, this is a measure for chromosomal instability and a high score (>0.128) is associated with poor prognosis. From Wurmbach et al. vascular invasion was used (Wang 2007, Iizuka 2003), from Lee et al. the different prognostic clusters that correlate with survival (cluster A with poor prognosis and cluster B with good prognosis) and from Chiang et al. the Barcelona Staging Classification (BCLC) (Llovet 1999). The Goeman-method was then applied for each individual prognostic factor in these data sets.

Microarray to Obtain a Chronic Hypoxia Gene Signature

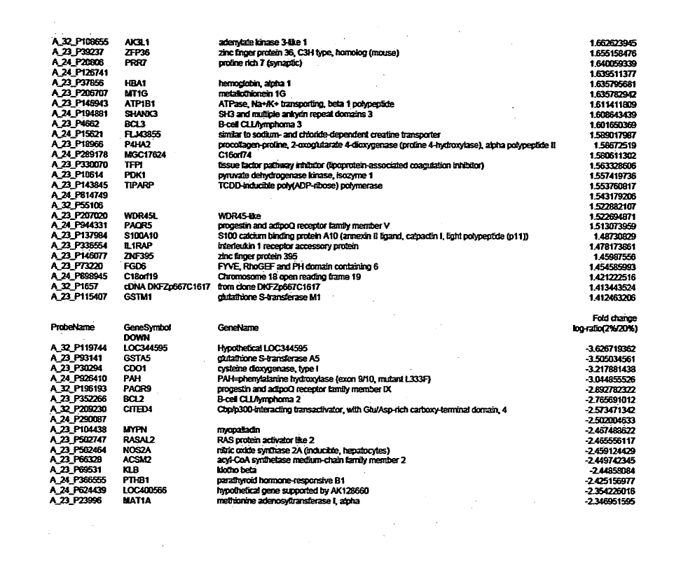

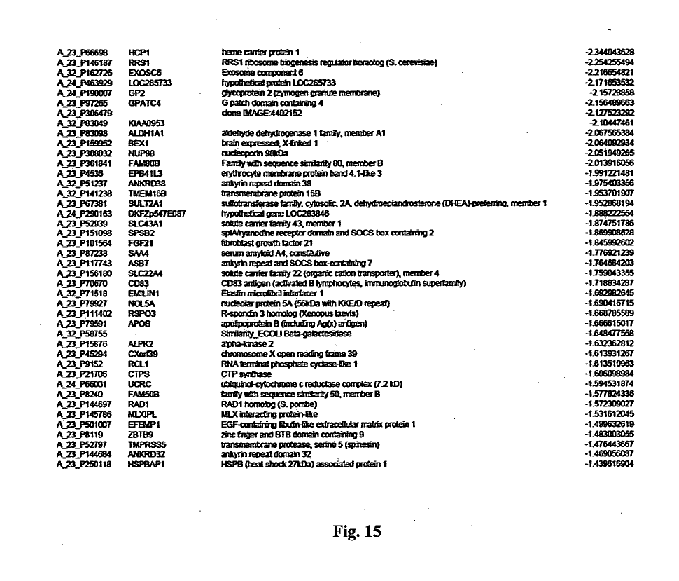

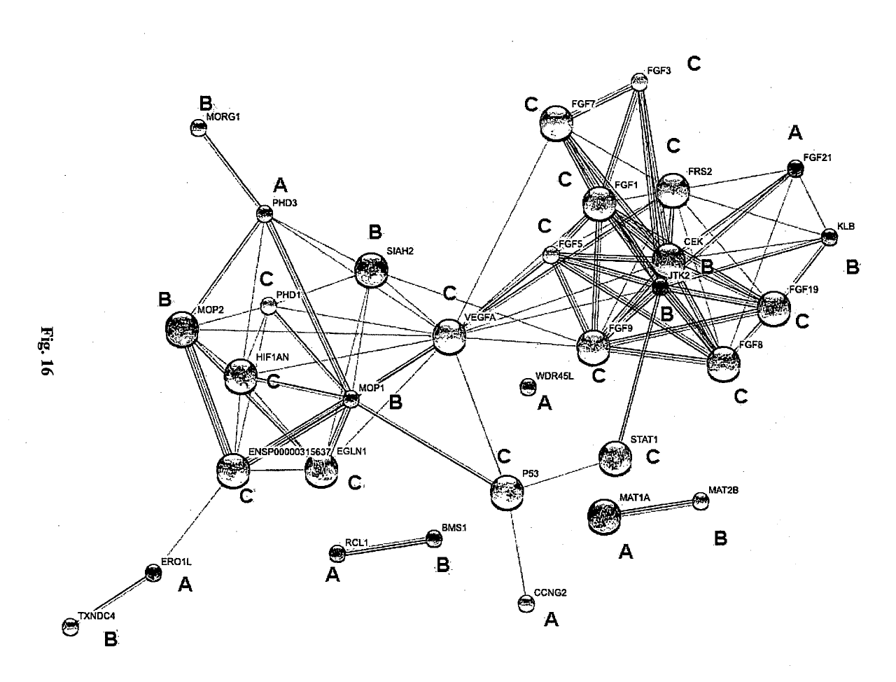

We started with the cell culture as model and determined the differentially expressed genes in HepG2 cells that were cultured for 72 hours at either 20% oxygen or in hypoxic conditions at 2% oxygen. We used the Agilent technology with colour flip on two independent experiments in duplicate resulting in 8 ratio values. To control the false discovery rate, multiple testing correction was performed and probes with a corrected p-value below 0.05 and a fold change of >2 were selected (Benjamini & Hochberg, 1995). A total of 37,707 spots showed a representative signal of which 2959 with a fold change above 2 and a corrected p-value <0.05. Selection of the highly significant genes (Limma correction p<0.01) resulted in 265 genes (207 upregulated and 58 downregulated, see FIG. 15), designated as the hypoxic gene set.

Analysis of Hypoxic Gene Expression in HCC Datasets

Our in vitro hypoxia gene set contains 265 genes, which we further investigated for clinical relevance. We used three published datasets to investigate the prognostic correlation and to optimize and reduce our hypoxia signature. The first three training datasets contained 229 HCCs and the validation dataset 91 HCCs. To test whether the overall expression pattern of these hypoxia genes is significantly related to the prognostic factor considered for each of the three training datasets, the global test of Goeman et al was used (Goeman, 2004). This resulted in a significant enrichment of the hypoxia gene set for all three training sets (p-value 0.03595 for Boyault, p-value <0.00001 for Lee and p-value 0.0064 for Wurmbach).

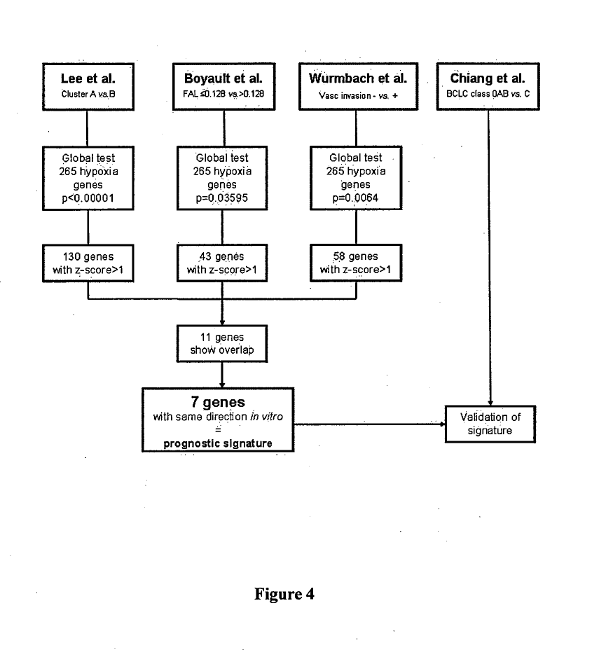

Next, when only keeping the significant genes with a z-score above 1, 130 genes remained for the dataset of Lee et al, 43 genes for Boyault et al, and 58 genes for Wurmbach et al. Finally, genes for which the direction of altered expression did not correspond to the direction observed in vivo were removed. With this approach, we were able to downsize our hypoxia gene set to seven genes, the hypoxia signature, found to overlap between the three training datasets (see FIG. 4).

In this hypoxia signature consisting of seven genes, four genes were upregulated and three downregulated (see table 5). For some of these genes, there is evidence for linkage to hypoxia, and others are important in the cell cycle (see discussion).

These genes were used to define a hypoxia score: Hypoxia-score=mean (expression ratio UP (log base 2))−mean (expression ratio DOWN (log base 2)). UP are the in vivo up-regulated genes (n=4) and DOWN the in vivo down-regulated genes (n=3). This score is then used to classify these patients. Finally, the Area under the Receiver Operating Characteristic (ROC) curve (AUC) curve was used to assess the predictive performance of the hypoxia-score in all data sets.

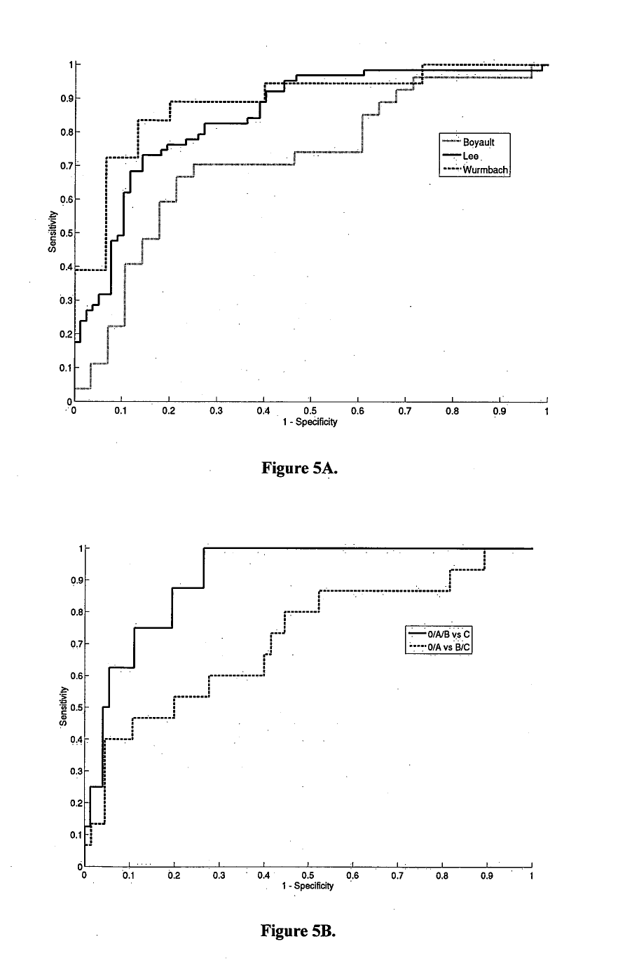

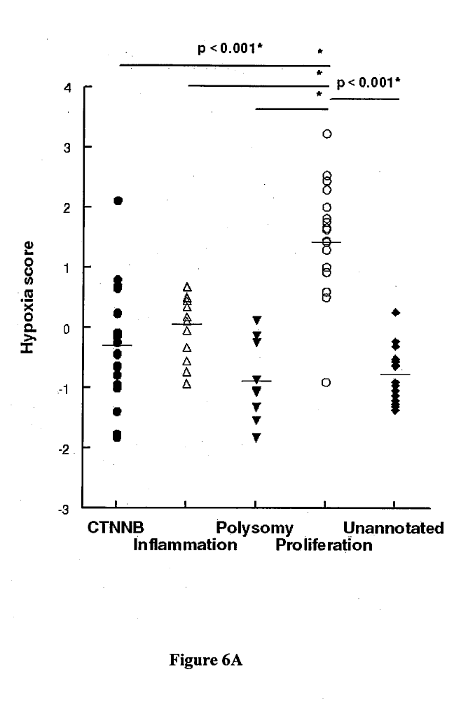

These seven genes could significantly divide patients with and without vascular invasion (Wurmbach, AUC 88.9%), with a FAL-index >0.128 and ≦0.128 (Boyault, AUC 72.8%) and with cluster A and cluster B gene expression (Lee, AUC 84.9%) (FIG. 5A). For validation, we used the Chiang dataset with the BCLC-classification as prognostic characteristic. The seven genes significantly separated the BCLC group 0/A/B and C (AUC 91%) (FIG. 5B), as well as the group 0/A and B/C (AUC 71.5%) (data not shown). Similar ROC curves were used to assess the predictive performance of particular subsets of the 7 hypoxia-related prognostic genes in HCC. The results are summarized in table 8a, 8b, 8c and 8d.

### Example 5

### Validation of the 7 Hypoxia-Related Prognostic Genes in HCC

Quantitative RT-PCR, Immunohistochemistry and Cell Metabolism

To confirm the microarray results we performed a new set of cell culture experiments on HepG2 cells at 20% O₂ and in parallel at 2% O₂. We analyzed the expression of selected genes at different time points (between 0 and 72 hours) by real-time PCR with each sample in duplicate. Real-time data at 72 hours are in agreement with microarray findings (table 3).

HIF1A showed a dynamic in its mRNA expression over time (FIG. 1) with an induction in the first phase and adaptation after longer exposure to reduced oxygen. Most of the other genes we investigated also showed a bi-phasic response. EGLN1, VEGF, IGFBP, ADM and LOX initially all went up and decline after they had peaked, FIH dropped in the first 24 hours and remained at that reduced level until the end of the experiment. CDO1 and BCL2 showed a gradual decrease over the whole time of the experiment. These observations support the initial assumption that the acute hypoxic state (up to 24 hrs) has a different gene expression pattern compared to the more chronic state. Immunohistochemical staining of HIF1A and VEGF in cultured cells showed a similar dynamic in time (FIGS. 2A and 2B).

Of the known hypoxia regulated genes all genes show dynamic behaviour, HIF1A is mainly active in the first 24-48 hours. In the chronic condition the expression returns almost back to baseline. The other genes also show dynamic changes under hypoxia, FIH is inhibited during hypoxia, while EGLN1 and VEGF show an upregulation (FIG. 1A). The five genes we selected for the confirmation of the results obtained by microarray (FIG. 1B) all showed at 72 hours similar expression by RT-PCR as obtained in our microarray experiment (table 3). Also for these genes, the long term hypoxia expression differs from that in the acute hypoxia situation.

Adaptation of the Metabolism to Chronic Exposure to Hypoxia.

The increase in XTT signal/100.000 cells (as determined by Coulter counter) after 4½ hours incubation was used as a measure for metabolic activity. The metabolic activity for cells cultured at 20% was set as reference at 100% (as demonstrated in table 4)

Determination of the metabolic activity of HepG2 cells immediately after exposure to 20% or 2% O₂ showed an increased activity in the cells that were exposed to low oxygen. No significant differences were found in the metabolic activity between cells that were grown at 20% or 2% O₂ for 72 hours. Cells in both cultures had the same metabolic activity per cell indicating that at this level the cells had adapted to chronic exposure to hypoxia.

Liver Specificity of 7-Gene Set

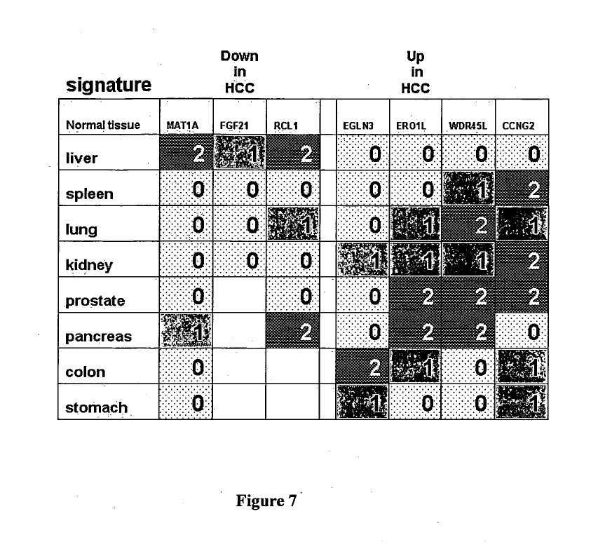

To determine the liver specificity of the 7-gene prognostic signature we retrieved expression data of normal human tissues from four data sets stored at NCBI. The data sets are: GDS422 and GDS423 (gene expression of a variety of normal tissue, with samples composed of a pool of 10-25 individuals), GDS 1209 (profiling normal human tissue samples obtained from 30 individuals) and GDS 1663 (normal tissue of 4 kidney, 4 liver, and 4 spleen, samples determined at two research centres). A semi-quantitative score was made based on the mean expression levels reported in the above mentioned four data sets. Expression values were classified into 4 groups: 0=<20%, 1=20-50%, 2=40-70% and 3=>70% (FIG. 7).

In normal liver tissue MAT1A, FGF21 and RCL1 are highly expressed which is not the case in other tissues for this combination of 3 genes. Because of their high expression under normoxic condition a downregulation of MAT1A, FGF21 and RCL1 under hypoxia will be distinguishable. The four other genes are low in expression in normal liver tissue and because they respond to hypoxia with increased expression any changes in their levels should also be detectable. Thus, none of the normal human tissues shows the same pattern for the 7 genes, making this set liver specific.

Example 7

### Survival and Early Recurrence

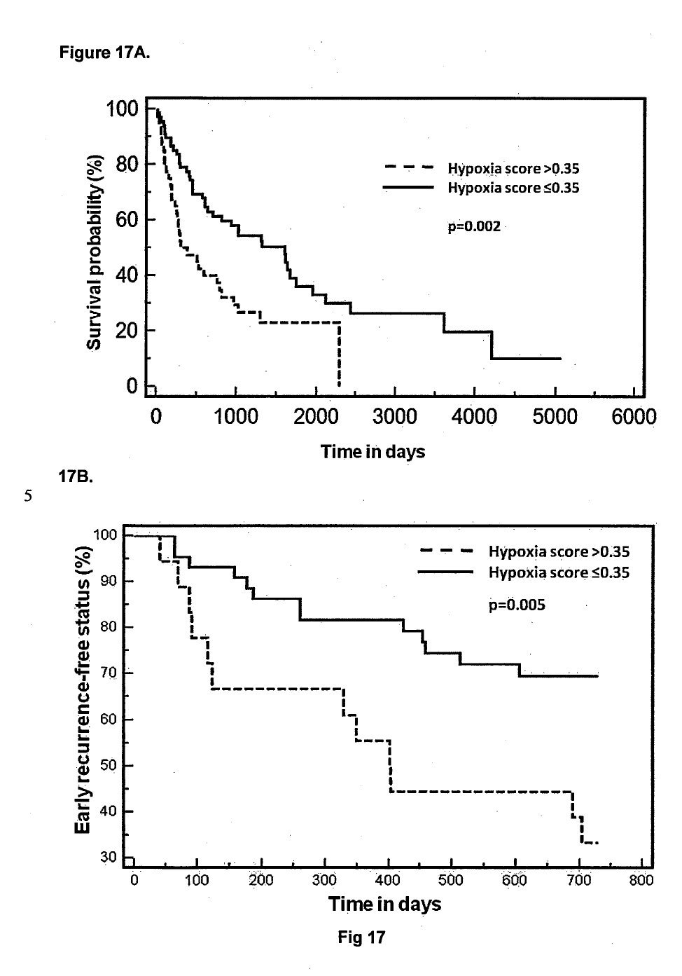

With the development of the hypoxia score we were able to test whether the score correlates with survival and recurrence. We conducted a retrospective survival analysis on 135 patients of the study by Lee et al. (MedCalc Software, version 11.0.1). We first determined the Cox proportional hazard ratio for survival, since our hypoxia score is a continuous variable. Indeed, the hypoxia score significantly increased the risk of death (HR 1.39, 95% CI 1.09-1.76, p=0.007). If we use a cut-off value of 0.35 for the hypoxia score (Log Rank test p=0.0018) we were able to demonstrate significant differences in survival in 135 patients with a Kaplan-Meier survival curve (FIG. 17A). The median survival for patients with a hypoxia score >0.35 (n=42) was 307 days, whereas the median survival for patients with a hypoxia score ≦0.35 (n=93) was 1602 days (p=0.002). For recurrence in HCC patients, it has been suggested to make a differentiation between early recurrence (<2 yrs) and late recurrence (>2 yrs). 27, 28 Early recurrence is the result of dissemination of the primary tumor and tumor characteristics determine the risk of recurrence. On the other hand, recurrence after 2 years is usually a second primary tumor that arises in a cirrhotic liver and has no relation with the first tumor. Risk of late recurrence is determined by clinical characteristics and they overlap with the general risk for HCC in cirrhotic patients. Since our hypoxia score is determined on the tumor tissue itself, we tested if it could predict early recurrence. We calculated a significant Cox proportional hazard ratio of 1.54 (95% CI=1.09-2.17, p=0.015), which means that with an elevation of the hypoxia score with 0.1 point, the risk of developing a recurrence is 5.4% higher. Again, when we use a cut-off of 0.35 for the hypoxia score, the Kaplan Meier curve shows a significant difference in early recurrence (p=0.005) (FIG. 17B).

By computational methods present invention identified 7 genes, out of 3592 differentially expressed under chronic hypoxia, that showed correlation with poor prognostic indicators in all training sets (272 patients) and this was validated in a 4th dataset (91 patients). The 7-gene set is associated with poor survival (HR 1.39, p=0.007) and early recurrence (HR 1.54, p=0.015). Retrospectively, using a hypoxia score based on this 7-gene set it was demonstrated that patients with a score >0.35 had a median survival of 307 days, whereas patients with a score ≦0.35 had a median survival of 1602 days (p=0.005).

Discussion

A general method for the classification and prediction of patient prognosis in HCC has not been possible to develop until now. Important to note is that HCC develops over many years and the process involves different kind of dysplastic changes that lead to malignancy. Which genes are affected depends on the underlying disease and the tumoral micro-environment. Recently, several studies have tried to identify gene sets with prognostic or diagnostic relevance by microarray analysis (Hoshida 2008). Each study resulted in its own classification with a specific separation into clusters. But, all these microarray studies show remarkable little overlap. The first major obstacle is the limited number of patients and different etiologies from which both clinical and corresponding molecular data are available. Furthermore, the results of the different studies seem to be centre dependent and related to the different microarray techniques used and also each study uses different clinical parameters for the evaluation and classification.

We started from the hypothesis that during cancer development the presence of hypoxia is a chronic situation which differs from acute hypoxia. Hypoxia is a well-known characteristic of solid tumours and has an established effect on the aggressiveness of tumours (Chan 2007, Gort 2008). It induces angiogenesis and anaerobic metabolism and promotes invasiveness (Sullivan 2007). To test our hypothesis independently of patient selection and variability, we decided to start from cell culture. Human liver cells HepG2 have detectible expression of 96% of the genes found in cultured primary hepatocytes (Harris 2004). And since our aim was to identify the effect of hypoxia on gene expression, we considered the microarray technique the best option to study the complete process.

In contrast to the previous studies on HCC we did not limit the number of genes we wanted to study by a priori selection, but used the Agilent 44 k microarray which covers all the known genes. Although the dynamics of gene expression indicate that after an adaptation period of 72 hours the gene expression is not as strongly altered as during the first 24 hours (FIG. 1), we still found that 8% of the genes were significantly changed at 72 hours.

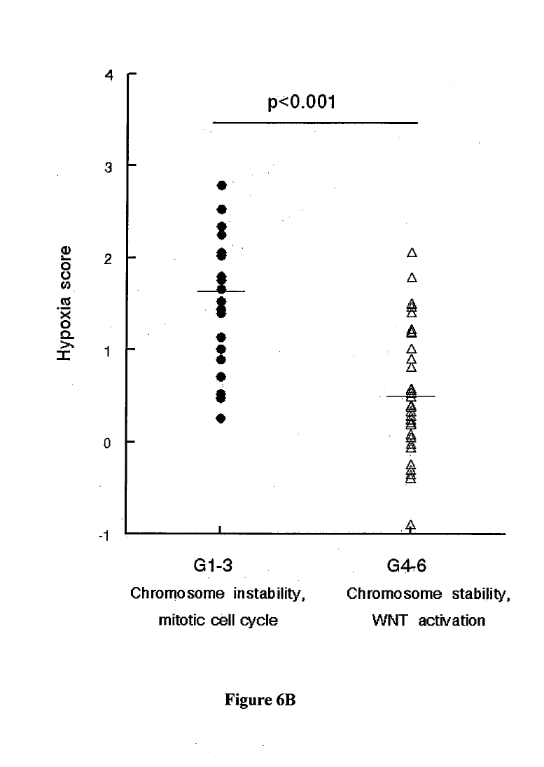

Starting with the group of 265 highly significant genes that came out of the microarray study of the HepG2 cells (table 3) we went through a sequence of analysis steps (FIG. 4) and compared the microarray data from 3 separate studies (Boyault 2007, Lee 2004, Lee 2006, Wurmbach 2007) with our group of genes. We could develop a very robust 7-gene prognostic signature using the method of Goeman et al. (Goeman 2004) (table 5. This seven gene prognostic set was applied to the fourth data set (Chiang 2008) and could significantly separate the BCLC group 0/A/B from C (FIG. 5B) or BCLC group 0/A from B/C (data not shown in graphics). Both in the study of Boyault et al as well as in the study by Chiang et al, the authors divided their patients into different subgroups. Using their classification we found that the hypoxia score corresponded with the subgroups that had the worse prognosis (FIGS. 6A and 6B).

When we compared the expression of the 7 genes in normal human tissues (FIG. 7), we found that the gene expression pattern for these genes in the liver is distinct from that found in other tissues. This makes the 7-gene set specific for classification of HCC.

The functions of these seven genes are either related to hypoxia, to cell cycle or to metabolism. Cyclin G2 (CCNG2) is an unconventional cyclin expressed at modest levels in proliferating cells, peaking during the late S and early G2-phase (Kasukabe 2008). It is significantly upregulated as cells exit the cell cycle in response to DNA damage. cDNA microarray analyses consistently point to CCNG2 upregulation in parallel with cell cycle inhibition during the responses to diverse growth inhibitory signals, such as heat shock, oxidative stress and hypoxia (Murray 2004). EGL nine homolog 3 (EGLN3), also prolyl hydroxylase 3, is a key regulator in chronic hypoxia. Recently it has been demonstrated that HIF1A is not overexpressed in chronic hypoxia due to upregulation of the different prolyl hydroxylases. In the acute phase EGLN1 has a dominant role, whereas EGLN3 comes into play during sustained hypoxia and promotes cell survival (Ginouves 2008), which supports our findings. ERO1-like (S. cerevisiae) (Ero1L) upregulation by hypoxia was demonstrated before in a variety of tumour cell lines, as well as in nontransformed, primary cells, including hepatocellular carcinoma cells (May 2005). In the first period (6 h) this is HIF dependent, but after 12 hrs there is also a HIF-independent manner (Gess 2003). ERO1L is necessary in the disulfide formation which is essential for the correct folding of proteins in the endoplasmic reticulum. Upregulation of ERO1L will proportionally increase the capability for proper protein folding under hypoxia in face of diminution in the ER oxidizing power due to the lack of oxygen and induces cell proliferation and survival. This response to hypoxia with upregulation of ERO1L is called the unfolded protein response (UPR) and regulates ER homeostasis and promotes hypoxia tolerance (Wouters 2008). WDR45L which encodes for a WD-40 repeat containing protein, is a member of a gene family involved in a variety of cellular processes, including cell cycle progression, signal transduction, apoptosis, and gene regulation. The exact function of WDR45L is unknown, but other family members such as WDR1 and WIPI3 are overexpressed in several human cancers (Proikas-Cezanne 2004). WDR16 is even overexpressed in a great majority of HCC patients and suppression leads to growth retardation (Pitella Silva 2005).

Fibroblast growth factor 21 (FGF21) is one of the downregulated genes in the hypoxia signature. FGF family members possess broad mitogenic and cell survival activities and are involved in a variety of biological processes including cell growth, tissue repair, tumour growth and invasion. The function of this particular growth factor has not yet been determined. Methionine adenosyltransferase 1 alpha (MAT1A) is critical for a differentiated and functional competent liver. It serves as a key enzyme in the production of S-adenosylmethionine, which is the source of methyl groups for most biological methylations (Mato 2002). In previous research it has been demonstrated that MAT1A is reduced in cirrhosis and HCC (Cai 1996, Avila 2000). Underexpression of MAT1A induces cell vulnerability to oxidative stress and facilitates the development to HCC (Martinez 2002). This gene is also underexpressed in the proliferation cluster of the two studies that published their molecular classification for HCC (Chiang and Boyault). RCL1 (RNA terminal phosphate cyclase-like 1) is also underexpressed in the proliferation cluster in both studies. The exact function of this cyclase in humans is not completely understood, but involves RNA pre-processing. In yeasts RCL1 is essential for viability and growth (Billy 2000).

The fact that both upregulated and downregulated genes are present in the same biological process such as the cell cycle underscores the complex biology of hypoxia in tumour cells. On the one hand hypoxia seems to induce growth retardation and inhibition of some metabolic processes, while on the other hand hypoxia favours uncontrolled growth, chemoresistance and cell survival.

To further explore the functional interactions or partnership between these 7 genes we loaded them into the STRING 8 program (http://string-db.org/). This program weights and integrates information from numerous sources, including experimental repositories, computational prediction methods and public text collections, thus acting as a meta-database that maps all interaction evidence onto a common set of genomes and proteins (Jensen et al. 2009). No direct link was found between the 7 genes. When we included 10 proven functional partners for said genes (e.g. MOP1=HIF1A) and 15 white nodes connecting hypoxia genes and the predicted functional partners (e.g. VEGFA) (see below table 6), it was found that 4 of the genes (EGLN3, ERO1L, CCNG2 and FGF21) are mapped within the hypoxia or hypoxix response cluster. The 3 other genes however (RCL1, MAT1A and WDR45L) were not mapped within the hypoxia or hypoxic response cluster, and the present study accordingly provides for the first time a functional link of these genes to hypoxia or hypoxic response. Perhaps these 3 genes represent the adaptation to prolonged hypoxia or a HIF/VEGF-independent regulation of gene expression.

Recently, the molecular classification of HCC has attracted a lot of attention. Based on gene expression patients can be classified to the beta-catenin subgroup, the proliferation subgroup, the inflammation subgroup or several others. The exact prognostic and therapeutic implications of this categorization is still unclear. In the study by Chiang et al. patients were divided into five subgroups (Beta-catenin, proliferation, inflammation, polysomy chromosome 7 and unannotated). We analyzed our hypoxia signature in the different subgroups and there was a clear correlation with the proliferation cluster (FIG. 6A). This cluster consists of genes related to the mTOR pathway and several cell cycle genes, such as cyclins. Our 7-prognostic gene set also contains several cell cycle related genes, and shows an important link with the mTOR pathway as well. This signalling pathway regulates cell growth, cell proliferation, protein transcription and survival by orchestrating several upstream signals. Recently, an important role for the mTOR pathway in HCC was demonstrated (Villanueva 2008). In addition, analysis of the pRPS6 staining in the subgroups as defined by Chiang et al (Chiang et al. 2008) showed a significant increase (indicating aberrant mTOR signaling) in the proliferation cluster (Table 7).

Multiple studies showed evidence for an interaction between mTOR and hypoxia (or HIF1). Several among them showed an oxygen independent induction of HIF1A by mTOR signalling, with an upregulation of several HIF targets such as VEGF (Zhong 2000, Land 2007). The upregulation of mTOR can be due to oncogenic mutations, for example in the PTEN gene. On the other hand the mTOR pathway is regulated by oxygen and nutrional signals (Arsham 2003). With oxygen and nutrient deprivation the mTOR pathway is inhibited and this influences tumour progression and hypoxia tolerance as well. In the early stage of cancer development this might lead to tumour suppression, however it is hypothesized that in the advanced stage of cancer development this can lead to hypoxia tolerance and inhibition of apoptosis (Wouters 2008). Multiple reasons can clarify the correlation between our hypoxia signature and the proliferation cluster. One can hypothesize that rapid proliferating cells suffer more extensively from hypoxia, since the neovascularization follows tumour expansion. Or it might be that although patients in the proliferation cluster show a hypoxic phenotype, this gene expression is purely based on upregulation of mTOR. This upregulation might lead to a hypoxia-like response with upregulation of HIF1A and further initiation of an adaptive response. Another explanation might be found in the fact that the chronic hypoxic phenotype is also under control of mTOR signalling. Hypoxia and mTOR are both key regulators of cellular metabolism and they show close relation to the endoplasmatic reticulum (ER) homeostasis.

In conclusion, our findings have potential implications in several areas:
1) We have demonstrated the involvement of chronic hypoxia in HCC development with prognostic value.
2) We identified a 7-gene prognostic signature that correlates with prognosis of the patient irrespectively from the array platform used and this signature can be used with different clinical criteria. Because our prognostic signature includes a limited set of 7 genes, this will make the application possible in different centres using real-time PCR techniques in stead of technically more advanced microarray analysis. As a prognostic factor it can have influence on the therapeutic options that are available for a patient. Therefore this signature needs to be validated in new prospective studies to demonstrate its use.
3) The method we used to identify this limited gene set, namely, the combination of a cell culture model and the global test method, can also be applied to other tumours. With this hypothesis driven method it is easier to extract the most important genes out of the large amount of information from the microarray technique. Furthermore, our approach has the big advantage that it combines different studies in a straight forward manner. In this way essential information can be extracted even when the number of patients that can be recruited into one study is limited, as with HCC patients.
4) We appreciate the value of hierarchic clustering of array data of patients and investigation of molecular classification of HCC. Here we demonstrate the added information that can be obtained from cell culture experiments. By starting from a clearly delimited hypothesis (chronic hypoxia) which led us to a small and pure data set we found clinical relevance.

Although in vitro studies are never fully representative for the situation as it develops in an organ, the validation in 4 clinical data sets proves the value of our study beyond theoretical objections.

Our findings have prognostic implications for HCC patients and therefore could be incorporated in the molecular classification of HCC.

### TABLES TO THIS DESCRIPTION

TABLE 1
List of genes and Affimetrix ID of RT-PCR assays used in this study.

| Gene | | | Assay ID |
|---|---|---|---|
| symbol | Gene Name | Chromosome | Affimetrix |
| ADM | Adrenomedullin | 11 | Hs00181605_m1 |
| B2M | Beta-2-microglobulin | 15 | Hs99999907_m1 |
| BCL2 | B-cell CLL/lymphoma 2 | 18 | Hs00236808_s1 |
| CDO1 | Cysteine dioxygenase, type I | 5 | Hs00156447_m1 |
| EGLN1 | Egl nine homolog 1 | 1 | Hs00254392_m1 |
| | (C. elegans) | | |
| HIF1A | Hypoxia-inducible factor | 14 | Hs00936368_m1 |
| | 1, alpha subunit | | |
| HIFAN | Hypoxia-inducible factor | 10 | Hs00215495_m1 |
| | 1 alpha inhibitor | | |
| IGFBP3 | Insulin-like growth factor | 7 | Hs00181211_m1 |
| | binding protein 3 | | |
| LOX | Lysyl oxidase | 5 | Hs00942480_m1 |
| VEGF-A | Vascular endothelial | 6 | Hs00173626_m1 |
| | growth factor A | | |

TABLE 2
Overview of published datasets that were used in this study.

| | Boyault | Lee | Wurmbach | Chiang |
|---|---|---|---|---|
| Dataset ID | E-TABM-36 | GSE1898 | GSE6764 | GSE9843 |
| | | GSE4024 | | |
| Array type | Affymetrix HG- | Human Array- | Affymetrix | Affymetrix |
| | U133A | Ready Oligo Set, | HG-U133A plus | HG-U133A plus |
| | | Qiagen | version 2.0 | version 2.0 |
| N array | 65 | 139 | 73 | 91 |
| N patients | 60 | 139 | 48 | 91 |
| N HCC | 57 | 140* | 33 | 91 |
| N control | 5 | 19 | 10 | ? |
| | Pools of samples | Pools of samples | | |
| N other | 3 | None | 30 | None |
| (cirrhosis, adenoma, | adenoma = 3 | | cirrhosis = 13, | |
| dysplasia) | | | dysplasia = 17 | |
| Sex | + | + | na | + |
| M/F | 47/13 | 102/37 | | 54/27 (na = 10) |
| Age | + | + | na | + |
| Mean age (yr) | 61 | 56 | | 65 (na = 10) |
| Underlying liver | +/− | + | + | + |
| disease | HBV status | 14 crypto, 16 (N)ASH, | All HCV | All HCV |
| | + = 15 | 56 HBV, 14 HCV, 5 | | |
| | | metabolic, 2 AIH, 1 | | |
| | | PBC, 9 combi, 22 na | | |
| Cirrhosis | na | + | + | na |
| | | 50% positive, na = 1 | All cirrhosis | |
| AFP | na | + | na | + |
| | | >300 = 55, | | >300 = 55, |
| | | na = 11 | | na = 22 |
| Tumour size | na | + | + | na |
| <5 cm > | | >5 = 77 na = 1 | | (BCLC)* |
| Differentiation | na | + | + | na |
| | | 1 = 2, 2 = 57, | 1 = 12, 2 = 9, | |
| | | 3 = 74, 4 =6 | 3 − 4 = 12, | |
| Vascular | na | + | + | na |
| invasion | | − = 21, + = 27, | no = 15, | (BCLC)* |
| | | na = 91 | mirco = 11, | |
| | | | macro = 7 | |
| Prognostic | na | + | na | na |
| clusters | | A = 60, B = 80 | | |
| Satellite | + | na | + | na |
| nodules** | 22/57 (39% +) | | 15/33 (45% +) | |
| BCLC score | na | na | na | + |
| | | | | 0 = 9, A = 56, |
| | | | | B = 7, C = 8, |
| | | | | na = 11 |
| FAL-index | + | na | na | na |
| | − = 29, + = 26, | | | |
| | na = 5 | | | |
| p53 mutation | + | na | na | + |
| | − = 45, + = 14, | | | − = 74, + = 11, |
| | na = 1 | | | na = 6 |
| Beta-catenin | + | na | na | + |
| mutation | − = 41, + = 18, | | | − = 60, + = 27, |
| | NA = 1 | | | NA = 4 |

*in the liver of one patient two separate HCC were found and these were analysed separately,
**Satellite nodules were defined differently in Boyault and Wurmbach.

TABLE 3
Comparison of gene expression ratio (²log) from microarray
and by RT-PCR for selected genes.

| | 2% vs 20% oxygen during 72 hours | |
|---|---|---|
| Gene | Array | PCR |
| CDO1 | −3.22 | −1.75 |
| BCL2 | −2.77 | −1.05 |
| LOX | 4.37 | 1.21 |
| ADM | 3.83 | 2.14 |
| IGFBP3 | 3.71 | 1.99 |
| HIF1A | 0.62 | 0.23 |
| VEGF | 2.51 | 2.25 |
| EGLN1 | 2.01 | 0.93 |

HepG2 cells were cultured for 72 hours in 2% O₂ or 20% O₂, cells were collected and after RNA extraction used in microarray or RT-PCR as described in materials and method.
The ratio between expression at 2% O₂ compared to that at 20% O₂ is presentedin the table.

TABLE 4
Response in metabolic activity to hypoxia.

| | 20% O₂ | 2% O₂ | p-value |
|---|---|---|---|
| Acute hypoxia | 100 ± 3.3% | 120.6 ± 4.9% | <0.001 |
| Chronic hypoxia | 100 ± 4.0% | 90.6 ± 10.2% | NS |

Metabolic activity defined as increased XTT conversion per 100.000 cells over 4 ½ hours was determined. Response of cells at 20% O₂ was set as 100%

TABLE 5
List of the 7 hypoxia-related prognostic genes in HCC.

| | | Response to |
|---|---|---|
| Gene | Full name | hypoxia |
| CCNG2 | Cyclin G2 | Upregulation |
| EGLN3 | Egl nine homolog 1 (C. elegans) | Upregulation |
| ERO1L | Endoplasmic Reticulum Oxidoreductin-1 L | Upregulation |
| FGF2I | Fibroblast growth factor 21 | Downregulation |
| MAT1A | Methionine adenosyltransferase I alpha | Downregulation |
| RCL1 | RNA terminal phosphate cyclase-like 1 | Downregulation |
| WDR45L | WDR45-like | Upregulation |

TABLE 6
List of the genes with their abbreviations and synonyms describing the protein
interactions using STRING 8.0 software.

| A | Input: 7 hypoxia related genes |
|---|---|
| FGF21 | Fibroblast growth factor 21 precursor (FGF-21) |
| PHD3 | Egl nine homolog 3 (EC 1.14.11.-) (EGLN3) (Hypoxia-inducible factor |
| | prolyl hydroxylase 3) (HIF-prolyl hydroxylase 3) (HIF-PH3) (HPH-1) |
| | (Prolyl hydroxylase domain-containing protein 3) (PHD3) |
| WDR45L | WD repeat domain phosphoinositide-interacting protein 3 (WIPI-3) (WD |
| | repeat protein 45-like) (WDR45-like protein) (WIPI49-like protein) |
| CCNG2 | Cyclin-G2 |
| ERO1L | ERO1-like protein alpha precursor (EC 1.8.4.-) (ERO1-Lalpha) |
| | (Oxidoreductin-1-Lalpha) (Endoplasmic oxidoreductin-1-like protein) |
| | (ERO1-L) |
| MAT1A | S-adenosylmethionine synthetase isoform type-1 (EC 2.5.1.6) (Methionine |
| | adenosyltransferase 1) (AdoMet synthetase 1) (Methionine |
| | adenosyltransferase MI) (MAT-I/III) |
| RCL1 | RNA 3′-terminal phosphate cyclase-like protein (Homo sapiens) |
| B | Predicted Functional Partners: |
| MOP1 | Hypoxia-inducible factor 1 alpha (HIF-1 alpha) (HIF1 alpha) (ARNT- |
| | interacting protein) (Member of PAS protein 1) (Basic-helix-loop- helix-PAS |
| | protein MOP1) |
| JTK2 | Fibroblast growth factor receptor 4 precursor (EC 2.7.10.1) (FGFR-4) |
| | (CD334) |
| KLB | Beta klotho (BetaKlotho) (Klotho beta-like protein) |
| BMS1 | Ribosome biogenesis protein BMS1 homolog |
| MOP2 | Endothelial PAS domain-containing protein 1 (EPAS-1) (Member of PAS |
| | protein 2) (Basic-helix-loop-helix-PAS protein MOP2) (Hypoxia-inducible |
| | factor 2 alpha) (HLF-2 alpha) (HIF2 alpha) (HIF-1 alpha-like factor) (HLF) |
| MORG1 | Mitogen-activated protein kinase organizer 1 (MAPK organizer 1) |
| TXNDC4 | Thioredoxin domain-containing protein 4 precursor (Endoplasmic reticulum |
| | resident protein ERp44) |
| MAT2B | methionine adenosyltransferase II, beta isoform 2 |
| CEK | Basic fibroblast growth factor receptor 1 precursor (EC 2.7.10.1) (FGFR-1) |
| | (bFGF-R) (Fms-like tyrosine kinase 2) (c-fgr) (CD331 antigen) |
| SIAH2 | E3 ubiquitin-protein ligase SIAH2 (EC 6.3.2.-) (Seven in absentia homolog |
| | 2) (Siah-2) (hSiah2) |
| C | White nodes, connecting hypoxia genes and predicted functional partners |
| FGF7 | Keratinocyte growth factor precursor (KGF) (Fibroblast growth factor 7) |
| | (FGF-7) (HBGF-7) |
| P53 | Cellular tumor antigen p53 (Tumor suppressor p53) (Phosphoprotein p53) |
| | (Antigen NY-CO-13) |
| FGF19 | Fibroblast growth factor 19 precursor (FGF-19) |
| HIF1AN | Hypoxia-inducible factor 1 alpha inhibitor (EC 1.14.11.16) (Hypoxia- |
| | inducible factor asparagine hydroxylase) (Factor inhibiting HIF-1) (FIH-1) |
| FRS2 | Fibroblast growth factor receptor substrate 2 (FGFR substrate 2) (Sucl- |
| | associated neurotrophic factor target 1) (SNT-1) |
| PHD1 | Egl nine homolog 2 (EC 1.14.11.-) (EGLN2) (Hypoxia-inducible factor |
| | prolyl hydroxylase 1) (HIF-prolyl hydroxylase 1) (HIF-PH1) (HPH-3) |
| | (Prolyl hydroxylase domain-containing protein 1) (PHD1) |
| FGF5 | Fibroblast growth factor 5 precursor (FGF-5) (HBGF-5) (Smag-82) |
| ENSP00000315637 | Aryl hydrocarbon receptor nuclear translocator (ARNT protein) (Hypoxia- |
| | inducible factor 1 beta) (HIF-1 beta) |
| FGF8 | Fibroblast growth factor 8 precursor (FGF-8) (HBGF-8) (Androgen- |
| | induced growth factor) (AIGF) |
| FGF3 | INT-2 proto-oncogene protein precursor (Fibroblast growth factor 3) |
| | (FGF-3) (HBGF-3) |
| FGF1 | Heparin-binding growth factor 1 precursor (HBGF-1) (Acidic fibroblast |
| | growth factor) (aFGF) (Beta-endothelial cell growth factor) (ECGF-beta) |
| EGLN1 | Egl nine homolog 1 (EC 1.14.11.-) (Hypoxia-inducible factor prolyl |
| | hydroxylase 2) (HIF-prolyl hydroxylase 2) (HIF-PH2) (HPH-2) (Prolyl |
| | hydroxylase domain-containing protein 2) (PHD2) (SM-20) |
| STAT1 | Signal transducer and activator of transcription 1-alpha/beta (Transcription |
| | factor ISGF-3 components p91/p84) |
| VEGFA | Vascular endothelial growth factor A precursor (VEGF-A) (Vascular |
| | permeability factor) (VPF) |
| FGF9 | Glia-activating factor precursor (GAF) (Fibroblast growth factor 9) (FGF- |
| | 9) (HBGF-9) |

A: The 7 hypoxia genes,
B: Predicted Functional Partners,
C: White nodes, connecting hypoxia genes and predicted functional partners

TABLE 7
Association of aberrant mTOR signaling in different
classes of HCC (from study by Chiang et al 2008).

| | p-RPS6 staining by immunohistochemistry | | | |
|---|---|---|---|---|
| Cluster | pos | neg | % pos | |
| CTNNB1 | 6 | 16 | 27.27 | |
| Proliferation | 18 | 5 | 78.26 | * |
| Interferon | 9 | 8 | 52.94 | |
| Polysomy chr7 | 2 | 7 | 22.22 | |
| Unannotated | 4 | 11 | 26.66 | |

Data reported here come from the supplementary material to the article in Cancer Res 2008. p-RPS6 phosphorylation, which is down-stream in the mTOR signaling pathway, was detected by immunohistochemistry. We calculated that mTOR signaling was significantly altered between the Proliferation cluster versus either CTNNB1-, Polysomy chr7-or Unannotated-cluster (* for Proliferation cluster vs either one of the three clusters mentioned, p < 0.001, Chi-square). Between other combination of clusters there was no significant difference.

TABLE 8
Table 8a Best models for each number of genes < 7

| | Mean AUC | | |
|---|---|---|---|
| | Performance | | |
| | (Boyault, Lee, | | |
| | Wurmbach) | Entrez Gene ID | Gene Name |
| 1 gene | 0.739 | 56270 | WDR45L |
| 2 genes | 0.795 | 56270, 4143 | WDR45L, MAT1A |
| 3 genes | 0.814 | 56270, 4143, 30001 | WDR45L, MAT1A, |
| | | | ERO1L |
| 4 genes | 0.821 | 56270, 4143, 30001, | WDR45L, MAT1A, |
| | | 10171 | ERO1L, RCL1 |
| 5 genes | 0.821 | 56270, 4143, 30001, | WDR45L, MAT1A, |
| | | 10171, 901 | ERO1L, RCL1, |
| | | | CCNG2 |
| 6 genes | 0.821 | 56270, 4143, 30001, | WDR45L, MAT1A, |
| | | 10171, 901, 112399 | ERO1L, RCL1, |
| | | | CCNG2, EGLN3 |
| 7 genes | 0.822 | 56270, 4143, 30001, | WDR45L, MAT1A, |
| | | 10171, 901, 112399, | ERO1L, RCL1, |
| | | 26291 | CCNG2, EGLN3, |
| | | | FGF21 |

Table 8b: Models including RCL1

| | Mean AUC | |
|---|---|---|
| | performance (Boyault, | |
| | Lee, Wurmbach) | Other genes |
| RCL1 | 0.723 | |
| RCL1 + best other gene | 0.785 | WDR45L |
| RCL1 + two best other genes | 0.804 | WDR45L, MAT1A |
| RCL1 + three best other genes | 0.821 | WDR45L, MAT1A, ERO1L |
| RCL1 + four best other genes | 0.821 | WDR45L, MAT1A, ERO1L, |
| | | CCNG2 |
| RCL1 + five best other genes | 0.821 | WDR45L, MAT1A, ERO1L, |
| | | CCNG2, EGLN3 |

Table 8c: Best models for genes not previously associated with HCC, i.e. WDR45L,
RCL1, CCNG2

| | Mean AUC | |
|---|---|---|
| | performance (Boyault, | |
| | Lee, Wurmbach) | Gene Name |
| All 3 genes | 0.798 | WDR45L, RCL1, |
| | | CCNG2 |
| Best 2/3 genes | 0.785 | WDR45L, RCL1 |
| Best 1/3 genes | 0.739 | WDR45L |

Table 8d: Best models for genes not previously associated with HCC, i.e. WDR45L,
RCL1, CCNG2 and one additional gene of the 7 hypoxia-related prognosticHCC genes

| | Mean AUC | |
|---|---|---|
| | performance (Boyault, | |
| | Lee, Wurmbach) | Gene Name |
| Best 3 unknown + | 0.810 | WDR45L, RCL1, |
| 1 known | | CCNG2, MAT |
| Best 2 unknown + | 0.804 | WDR45L, RCL1 , |
| 1 known | | MAT1A |
| Best 1 unknown + | 0.795 | WDR45L, MAT1A |
| 1 known | | |

### REFERENCES TO THIS APPLICATION

Alqawi, 0., H. P. Wang, et al. (2007). “Chronic hypoxia promotes an aggressive phenotype in rat prostate cancer cells.” Free Radic Res 41(7): 788-97.
Arsham, A. M., J. J. Howell, et al. (2003). “A novel hypoxia-inducible factor-independent hypoxic response regulating mammalian target of rapamycin and its targets.” J Biol Chem 278(32): 29655-60.
Avila, M. A., C. Berasain, et al. (2000). “Reduced mRNA abundance of the main enzymes involved in methionine metabolism in human liver cirrhosis and hepatocellular carcinoma.” J Hepatol 33(6): 907-14.
Benjamini Y., Hochberg Y. (1995) “Controlling the false discovery rate: a practical and powerful approach to multiple testing.” J. Roy. Stat. Soc. B. 57:289-300:
Billy, E., T. Wegierski, et al. (2000). “Rcl1p, the yeast protein similar to the RNA 3′-phosphate cyclase, associates with U3 snoRNP and is required for 18S rRNA biogenesis.” Embo J 19(9): 2115-26.
Boyault, S., D. S. Rickman, et al. (2007). “Transcriptome classification of HCC is related to gene alterations and to new therapeutic targets.” Hepatology 45(1): 42-52.
Brahimi-Horn, M. C., J. Chiche, et al. (2007). “Hypoxia and cancer.” J Mol Med 85(12): 1301-7.
Brown, J. M. and A. J. Giaccia (1998). “The unique physiology of solid tumours: opportunities (and problems) for cancer therapy.” Cancer Res 58(7): 1408-16.
Brown, J. M. and W. R. Wilson (2004). “Exploiting tumour hypoxia in cancer treatment.” Nat Rev Cancer 4(6): 437-47.
Budhu, A., M. Forgues, et al. (2006). “Prediction of venous metastases, recurrence, and prognosis in hepatocellular carcinoma based on a unique immune response signature of the liver microenvironment.” Cancer Cell 10(2): 99-111.
Cai, J., W. M. Sun, et al. (1996). “Changes in S-adenosylmethionine synthetase in human liver cancer: molecular characterization and significance.” Hepatology 24(5): 1090-7.
Chan D A, Giaccia A J. (2007) “Hypoxia, gene expression, and metastasis.” Cancer Metastasis Rev. 26(2):333-9.
Chang, H. Y., D. S. Nuyten, et al. (2005). “Robustness, scalability, and integration of a wound-response gene expression signature in predicting breast cancer survival.” Proc Natl Acad Sci USA 102(10): 3738-43.
Chen, X., S. T. Cheung, et al. (2002). “Gene expression patterns in human liver cancers.” Mol Biol Cell 13(6): 1929-39.
Chiang, D. Y., A. Villanueva, et al. (2008). “Focal gains of VEGFA and molecular classification of hepatocellular carcinoma.” Cancer Res 68(16): 6779-88.
Detre, S., G. Saclani Jotti, et al. (1995). “A “quickscore” method for immunohistochemical semiquantitation: validation for oestrogen receptor in breast carcinomas.” J Clin Pathol 48(9): 876-8.
Dvorchik, I., M. Schwartz, et al. (2008). “Fractional allelic imbalance could allow for the development of an equitable transplant selection policy for patients with hepatocellular carcinoma.” Liver Transpl 14(4): 443-50.
Edgar R, Domrachev M, Lash A E. (2002) “Gene Expression Omnibus: NCBI gene expression and hybridization array data repository.” Nucleic Acids Res. 30(1):207-10
Ein-Dor, L., O. Zuk, et al. (2006). “Thousands of samples are needed to generate a robust gene list for predicting outcome in cancer.” Proc Natl Acad Sci USA 103(15): 5923-8.
Fattovich, G., G. Giustina, et al. (1997). “Morbidity and mortality in compensated cirrhosis type C: a retrospective follow-up study of 384 patients.” Gastroenterology 112(2): 463-72.
Fink, T., P. Ebbesen, et al. (2001). “Quantitative gene expression profiles of human liver-derived cell lines exposed to moderate hypoxia.” Cell Physiol Biochem 11(2): 105-14.
Folkman, J., P. Hahnfeldt, et al. (2000). “Cancer: looking outside the genome.” Nat Rev Mol Cell Biol 1(1): 76-9.
Fortina P, Surrey S. (2008) “Digital mRNA profiling.” Nat Biotechnol. 26(3):293-4.
Gentleman, R. C., V. J. Carey, et al. (2004). “Bioconductor: open software development for computational biology and bioinformatics.” Genome Biol 5(10): R80.
Gess, B., K. H. Hofbauer, et al. (2003). “The cellular oxygen tension regulates expression of the endoplasmic oxidoreductase ERO1-L alpha.” Eur J Biochem 270(10): 2228-35.
Ginouves, A., K. Ilc, et al. (2008). “PHDs overactivation during chronic hypoxia “desensitizes” HIFalpha and protects cells from necrosis.” Proc Natl Acad Sci USA 105(12): 4745-50.
Goeman, J. J., S. A. van de Geer, et al. (2004). “A global test for groups of genes: testing association with a clinical outcome.” Bioinformatics 20(1): 93-9.
Gort, E. H., A. J. Groot, et al. (2008). “Hypoxic regulation of metastasis via hypoxia-inducible factors.” Curr Mol Med 8(1): 60-7.
Harris, A. J., S. L. Dial, et al. (2004). “Comparison of basal gene expression profiles and effects of hepatocarcinogens on gene expression in cultured primary human hepatocytes and HepG2 cells.” Mutat Res 549(1-2): 79-99.
Holmquist-Mengelbier, L., E. Fredlund, et al. (2006). “Recruitment of HIF-1alpha and HIF-2alpha to common target genes is differentially regulated in neuroblastoma: HIF-2alpha promotes an aggressive phenotype.” Cancer Cell 10(5): 413-23.
Hoshida, Y., A. Villanueva, et al. (2008). “Gene Expression in Fixed Tissues and Outcome in Hepatocellular Carcinoma. ” N Engl J Med. 359(19):1995-2004.
Iizuka, N., M. Oka, et al. (2003). “Oligonucleotide microarray for prediction of early intrahepatic recurrence of hepatocellular carcinoma after curative resection.” Lancet 361(9361): 923-9.
Jensen L J, et al. (2009). “ STRING 8—a global view on proteins and their functional interactions in 630 organisms.” Nucleic Acids Res. Jan(37):D412-6.
Kasukabe, T., J. Okabe-Kado, et al. (2008). “Cotylenin A, a new differentiation inducer, and rapamycin cooperatively inhibit growth of cancer cells through induction of cyclin G2.” Cancer Sci 99(8): 1693-8.
Kim, K. R., H. E. Moon, et al. (2002). “Hypoxia-induced angiogenesis in human hepatocellular carcinoma.” J Mol Med 80(11): 703-14.
Land, S. C. and A. R. Tee (2007). “Hypoxia-inducible factor 1alpha is regulated by the mammalian target of rapamycin (mTOR) via an mTOR signaling motif.” J Biol Chem 282(28): 20534-43.
Lee, J. S., I. S. Chu, et al. (2004). “Classification and prediction of survival in hepatocellular carcinoma by gene expression profiling.” Hepatology 40(3): 667-76.
Lee, J. S., J. Heo, et al. (2006). “A novel prognostic subtype of human hepatocellular carcinoma derived from hepatic progenitor cells.” Nat Med 12(4): 410-6.
Liu, E. T. (2005). “Mechanism-derived gene expression signatures and predictive biomarkers in clinical oncology.” Proc Natl Acad Sci USA 102(10): 3531-2.
Llovet, J. M., C. Bru, et al. (1999). “Prognosis of hepatocellular carcinoma: the BCLC staging classification.” Semin Liver Dis 19(3): 329-38.
Llovet, J. M., A. Burroughs, et al. (2003). “Hepatocellular carcinoma.” Lancet 362(9399): 1907-17.
Martinez-Chantar, M. L., F. J. Corrales, et al. (2002). “Spontaneous oxidative stress and liver tumours in mice lacking methionine adenosyltransferase 1A.” Faseb J 16(10): 1292-4.
Mato, J. M., F. J. Corrales, et al. (2002). “S-Adenosylmethionine: a control switch that regulates liver function.” Faseb J 16(1): 15-26.
May, D., A. Itin, et al. (2005). “Ero1-L alpha plays a key role in a HIF-1-mediated pathway to improve disulfide bond formation and VEGF secretion under hypoxia: implication for cancer.” Oncogene 24(6): 1011-20.
Murray, J. I., M. L. Whitfield, et al. (2004). “Diverse and specific gene expression responses to stresses in cultured human cells.” Mol Biol Cell 15(5): 2361-74.
Parkin, D. M., F. Bray, et al. (2005). “Global cancer statistics, 2002.” CA Cancer J Clin 55(2): 74-108.
Parkinson H, Kapushesky M, et al. (2009) ArrayExpress update—from an archive of functional genomics experiments to the atlas of gene expression. Nucleic Acids Res. 2009 Jan;37(Database issue):D868-72. PubMed PMID: 19015125.
Proikas-Cezanne, T., S. Waddell, et al. (2004). “WIPI-1alpha (WIPI49), a member of the novel 7-bladed WIPI protein family, is aberrantly expressed in human cancer and is linked to starvation-induced autophagy.” Oncogene 23(58): 9314-25.
Semenza, G. L. (2003). “Targeting HIF-1 for cancer therapy.” Nat Rev Cancer 3(10): 721-32.
Silva, F. P., R. Hamamoto, et al. (2005). “WDRPUH, a novel WD-repeat-containing protein, is highly expressed in human hepatocellular carcinoma and involved in cell proliferation.” Neoplasia 7(4): 348-55.
Smyth, G. K. (2004). “Linear models and empirical bayes methods for assessing differential expression in microarray experiments.” Stat Appl Genet Mol Biol 3: Article3.
Sonna, L. A., M. L. Cullivan, et al. (2003). “Effect of hypoxia on gene expression by human hepatocytes (HepG2).” Physiol Genomics 12(3): 195-207.
Sullivan R, Graham C H. (2007) “Hypoxia-driven selection of the metastatic phenotype.” Cancer Metastasis Rev. 26(2):319-31.
Thorgeirsson, S. S., J. S. Lee, et al. (2006). “Functional genomics of hepatocellular carcinoma.” Hepatology 43(2 Suppl 1): S145-50.
Tian, L., S. A. Greenberg, et al. (2005). “Discovering statistically significant pathways in expression profiling studies.” Proc Natl Acad Sci USA 102(38): 13544-9.
Vengellur, A., J. M. Phillips, et al. (2005). “Gene expression profiling of hypoxia signaling in human hepatocellular carcinoma cells.” Physiol Genomics 22(3): 308-18.
Villanueva, A., et al. (2008). “ Pivotal role of mTOR signaling in hepatocellular carcinoma.” Gastroenterology. 135(6):1972-83.
Wang, S. M., L. L. Ooi, et al. (2007). “Identification and validation of a novel gene signature associated with the recurrence of human hepatocellular carcinoma.” Clin Cancer Res 13(21): 6275-83.
Wouters, B. G. and M. Koritzinsky (2008). “Hypoxia signalling through mTOR and the unfolded protein response in cancer.” Nat Rev Cancer 8(11): 851-64.
Wurmbach, E., Y. B. Chen, et al. (2007). “Genome-wide molecular profiles of HCV-induced dysplasia and hepatocellular carcinoma.” Hepatology 45(4): 938-47.
Yeh, S. H., P. J. Chen, et al. (2001). “Chromosomal allelic imbalance evolving from liver cirrhosis to hepatocellular carcinoma.” Gastroenterology 121(3): 699-709.
Zhong, H., K. Chiles, et al. (2000). “Modulation of hypoxia-inducible factor 1alpha expression by the epidermal growth factor/phosphatidylinositol 3-kinase/PTEN/AKT/FRAP pathway in human prostate cancer cells: implications for tumour angiogenesis and therapeutics.” Cancer Res 60(6): 1541-5.

1. An in vitro method for predicting or determining biological behaviour or a stage of a HCC tumour comprising:
determining the level of gene expression of at least three genes selected from the group consisting of CCNG2, EGLN3, ERO1L, FGF21, MAT1A, RCL1 and WDR45L, or a substantially similar marker for CCNG2, EGLN3, ERO1L, FGF21, MAT1 A, RCL1 or WDR45L in an isolated sample; and
comparing said levels of gene expression to a control; wherein a change in expression levels when compared to said control is indicative for the biological behaviour or a stage of HCC tumours.

2. The in vitro method according to claim 1, wherein the level of gene expression is determined from genes selected from the group consisting of CCNG2, EGLN3, ERO1L, FGF21, MAT1A, RCL1 and WDR45L.

3. The in vitro method according to claim 1 wherein one of the genes comprises RCL1 and wherein the other genes are selected from the group consisting of WDR45L, MAT1 A, ERO1L, CCNG2 and EGLN3.

4. The in vitro method according to claim 1 comprising determining the level of gene expression of RCL1, WDR45L and MAT1A.

5. The in vitro method according to claim 1 wherein the amount of increase in expression level of at least one of WDR45L, CCNG2, EGLN3 and ERO1L; and/or the amount of decrease in expression level of at least one of RCL1, MAT1A, and FGF21 is indicative of increased severity or invasiveness of the HCC tumour.

6. The in vitro method according to claim 1 wherein the amount of increase in expression level of at least one of WDR45L, CCNG2, EGLN3 and ERO1L; and/or the amount of decrease in expression level of at least one of RCL1, MAT1A, and FGF21 is indicative of increased proliferation of the HCC tumour.

7. The in vitro method according to claim 1 wherein the amount of increase in expression level of at least one of WDR45L, CCNG2, EGLN3 and ERO1L; and/or the amount of decrease in expression level of at least one of RCL1, MAT1A, and FGF21 is indicative of increased morbidity of the HCC tumour.

8. The in vitro method according to claim 1 wherein the amount of increase in expression level of at least one of WDR45L, CCNG2, EGLN3 and ERO1L; and/or the amount of decrease in expression level of at least one of RCL1, MAT1A, and FGF21 is indicative of an increased risk of mortality of the patient.

9. The in vitro method according to claim 1, wherein the level of gene expression is determined using one or more oligonucleotides specific for a gene selected from the group consisting of CCNG2, EGLN3, ERO1L, FGF21, MAT1A, RCL1 and WDR45L.

10. A kit for predicting or determining biological behaviour or a stage of a HCC tumour comprising a means for determining the level of gene expression of at least three genes selected from the group consisting of CCNG2, EGLN3, ERO1L, FGF21, MAT1A, RCL1 and WDR45L.

11. The kit according to claim 10 wherein one of the at least three genes comprises RCL1.

12. The kit according to claim 11, wherein the other genes are selected from the group consisting of WDR45L, MAT1 A, ERO1L, CCNG2 and EGLN3.

13. The kit of claim 10 wherein the means for determining the level of gene expression comprises one or more oligonucleotides specific for a marker gene selected of the group consisting of CCNG2, EGLN3, ERO1L, FGF21, MAT1A, RCL1 and WDR45L.

14. The kit according to claim 10 wherein the means for determining the level of gene expression comprises methods selected from Northern blot analysis, reverse transcription PCR or real time quantitative PCR, branched DNA, nucleic acid sequence based amplification (NASBA), transcription-mediated amplification, ribonuclease protection assay, and microarrays.

15. The kit according to claim 10 wherein the means for determining the level of gene expression comprises at least one antibody specific for a protein encoded by the marker gene selected from the group consisting of EGLN3, ERO1L, FGF21, MAT1A, WDR45L and CCNG2.

16. The kit according to claim 15 wherein the antibody is selected from the group consisting of polyclonal antibodies, monoclonal antibodies, humanized or chimeric antibodies, and biologically functional antibody fragments sufficient for binding of the antibody fragment to the EGLN3, ERO1L, FGF21, MAT1A, WDR45L and CCNG2 markers or substantially similar markers.

17. The kit according to claim 15 wherein the means for determining the level of gene expression comprises an immunoassay method.


SEQUENCE LISTING (14 sequences)

SEQ ID NO: 1 - DNA, 2074 bp, Homo sapiens
ccacgcgtccgggctcggggaggtttccgctgaggcggcgggggtgcggcggtgggctggtcttccgcggccggcgttgcgccgcggcggagggtgggcgcgcggggagcgggatggagccggggctgtgaggccgaggcggcggtgcctgggaggaagggtcggatgccggaccgggggcaccgctgaggcggtgggtccccgacctgcgagacaggtttggaagcccccgctgcgcccagtccgtgcggaccgcgaggtcgcgggcgggtggaggcgcgtctccggcacgatgaaggatttgggggcagagcacttggcaggtcatgaaggggtccaacttctcgggttgttgaacgtctacctggaacaagaagagagattccaacctcgagaaaaagggctgagtttgattgaggctaccccggagaatgataacactttgtgtccaggattgagaaatgccaaagttgaagatttaaggagtttagccaacttttttggatcttgcactgaaacttttgtcctggctgtcaatattttggacaggttcttggctcttatgaaggtgaaacctaaacatttgtcttgcattggagtctgttcttttttgctggctgctagaatagttgaagaagactgcaatattccatccactcatgatgtgatccggattagtcagtgtaaatgtactgcttctgacataaaacggatggaaaaaataatttcagaaaaattgcactatgaattggaagctactactgccttaaactttttgcacttataccatactattatactttgtcatacttcagaaaggaaagaaatactgagccttgataaactagaagctcagctgaaagcttgcaactgccgactcatcttttcaaaagcaaaaccatctgtattagccttgtgccttctcaatttggaagtggaaactttgaaatctgttgaattactggaaattctcttgctagttaaaaaacattccaagattaatgacactgagttcttctactggagagagttggtttctaaatgcctagccgagtattcttctcctgaatgttgcaaaccagatcttaagaagttggtttggatcgtttcaaggcgcacagcccagaacctccacaacagctactatagtgttcctgagctgccaacgatacctgaggggggttgttttgatgaaagtgaaagctctgttgcccaggctggagtgcagtggcccgatctcagctcattccaacctccacctaccaggttcaagcgattctcatgcctcagcctccggagtagctgggattacagctcctggacctgaagcagacctgcagagaggatcaagtgcagagacaagctgctgctgagatcatcataggggacctcttctaacatatgagttcctgaggaagtgctgtgaccttccaatgctgccactggtagtctcatttcaaagagtaaaatgtgaaaatccaatggcagcagctattgtgatatgccagacagcaggaattgggactaaaaatcaagggatacttgtgataatagatccatagtactttggagctggaagaaacccagcttcaaaaaacagcttgatttgcagctcttcattcaaaaagaatcccatccagggtatgacattgaacttactcatcacgtctctatagtgtcttctggagtttggaactaagacccctagacagcagagcctaagggaggaatgactctaccaggagacaaaataatgactctgttaaagttgagatggccggctgcctcatacctctgattcaaaagtaaaggagttgctgtactggttagggcgattgttcctgacaaccaaggggaaactggactgctacacaaggaggactgaacagagctgaggcttccctaatgaagacattctgctgtggtcagaagtttcagcagcccctgagttcctgcctgttcctcctgaaagccttccctatctgtggataatgggcttgccttgccaacttccacaattgcataaaacaagtccttgcaatgaatctcaacatgtattaaaaaaaaaaaaaaa

SEQ ID NO: 2 - protein, 344 aa, Homo Sapiens
MKDLGAEHLAGHEGVQLLGLLNVYLEQEERFQPREKGLSLIEATPENDNTLCPGLRNAKVEDLRSLANFFGSCTETFVLAVNILDRFLALMKVKPKHLSCIGVCSFLLAARIVEEDCNIPSTHDVIRISQCKCTASDIKRMEKIISEKLHYELEATTALNFLHLYHTIILCHTSERKEILSLDKLEAQLKACNCRLIFSKAKPSVLALCLLNLEVETLKSVELLEILLLVKKHSKINDTEFFYWRELVSKCLAEYSSPECCKPDLKKLVWIVSRRTAQNLHNSYYSVPELPTIPEGGCFDESESSVAQAGVQWPDLSSFQPPPTRFKRFSCLSLRSSWDYSSWT

SEQ ID NO: 3 - DNA, 2722 bp, Home Sapiens
gagtctggccgcagtcgcggcagtggtggcttcccatccccaaaaggcgccctccgactccttgcgccgcactgctcgccgggccagtccggaaacgggtcgtggagctccgcaccactcccgctggttcccgaaggcagatcccttctcccgagagttgcgagaaactttcccttgtccccgacgctgcagcggctcgggtaccgtggcagccgcaggtttctgaaccccgggccacgctccccgcgcctcggcttcgcgctcgtgtagatcgttccctctctggttgcacgctggggatcccggacctcgattctgcgggcgagatgcccctgggacacatcatgaggctggacctggagaaaattgccctggagtacatcgtgccctgtctgcacgaggtgggcttctgctacctggacaacttcctgggcgaggtggtgggcgactgcgtcctggagcgcgtcaagcagctgcactgcaccggggccctgcgggacggccagctggcggggccgcgcgccggcgtctccaagcgacacctgcggggcgaccagatcacgtggatcgggggcaacgaggagggctgcgaggccatcagcttcctcctgtccctcatcgacaggctggtcctctactgcgggagccggctgggcaaatactacgtcaaggagaggtctaaggcaatggtggcttgctatccgggaaatggaacaggttatgttcgccacgtggacaaccccaacggtgatggtcgctgcatcacctgcatctactatctgaacaagaattgggatgccaagctacatggtgggatcctgcggatatttccagaggggaaatcattcatagcagatgtggagcccatttttgacagactcctgttcttctggtcagatcgtaggaacccacacgaagtgcagccctcttacgcaaccagatatgctatgactgtctggtactttgatgctgaagaaagggcagaagccaaaaagaaattcaggaatttaactaggaaaactgaatctgccctcactgaagactgaccgtgctctgaaatctgctggccttgttcattttagtaacggttcctgaattctcttaaattctttgagatccaaagatggcctcttcagtgacaacaatctccctgctacttcttgcatccttcacatccctgtcttgtgtgtggtacttcatgttttcttgccaagactgtgttgatcttcagatactctctttgccagatgaagttacttgctaactccagaaattcctgcagacatcctactcggccagcggtttacctgatagattcggtaatactatcaagagaagagcctaggagcacagcgagggaatgaaccttacttgcactttatgtatacttcctgatttgaaaggaggaggtttgaaaagaaaaaaatggaggtggtagatgccacagagaggcatcacggaagccttaacagcaggaaacagagaaatttgtgtcatctgaacaatttccagatgttcttaatccagggctgttggggtttctggagaattatcacaacctaatgacattaatacctctagaaagggctgctgtcatagtgaacaatttataagtgtcccatggggcagacactccttttttcccagtcctgcaacctggattttctgcctcagccccattttgctgaaaataatgactttctgaataaagatggcaacacaattttttctccattttcagttcttacctgggaacctaattccccagaagctaaaaaactagacattagttgttttggttgctttgttggaatggaatttaaatttaaatgaaaggaaaaatatatccctggtagttttgtgttaaccactgataactgtggaaagagctaggtctactgatatacaataaacatgtgtgcatcttgaacaatttgagaggggaggtggagttggaaatgtgggtgttcctgttttttttttttttttttttttagttttcctttttaatgagctcaccctttaacacaaaaaaagcaaggtgatgtattttaaaaaaggaagtggaaataaaaaaatctcaaagctatttgagttctcgtctgtccctagcagtctttcttcagctcacttggctctctagatccactgtggttggcagtatgaccagaatcatggaatttgctagaactgtggaagcttctactcctgcagtaagcacagatcgcactgcctcaataacttggtattgagcacgtattttgcaaaagctacttttcctagttttcagtattactttcatgttttaaaaatccctttaatttcttgcttgaaaatcccatgaacattaaagagccagaaatattttcctttgttatgtacggatatatatatatatagtcttccaagatagaagtttactttttcctcttctggttttggaaaatttccagataagacatgtcaccattaattctcaacgactgctctattttgttgtacggtaatagttatcaccttctaaattactatgtaatttattcacttattatgtttattgtcttgtatcctttctctggagtgtaagcacaatgaagacaggaattttgtatatttttaaccaatgcaacatactctcagcacctaaaatagtgccgggaacatagtaagggctcagtaaatacttgttgaataaactcagtctcctacattagcattctaa

SEQ ID NO: 4 - protein, 239 aa, Homo Sapiens
MPLGHIMRLDLEKIALEYIVPCLHEVGFCYLDNFLGEVVGDCVLERVKQLHCTGALRDGQLAGPRAGVSKRHLRGDQITWIGGNEEGCEAISFLLSLIDRLVLYCGSRLGKYYVKERSKAMVACYPGNGTGYVRHVDNPNGDGRCITCIYYLNKNWDAKLHGGILRIFPEGKSFIADVEPIFDRLLFFWSDRRNPHEVQPSYATRYAMTVWYFDAEERAEAKKKFRNLTRKTESALTED

SEQ ID NO: 5 - DNA, 3334 bp, Homo sapiens
gcacgagccccgggctgccggcgcgggcgccgcggcacgtccacaggctgggtcgcgaggtggcgatcgctgagaggcaggagggccgaggcgggcctgggaggcggcccggaggtggggcgccgctggggccggcccgcacgggcttcatctgagggcgcacggcccgcgaccgagcgtgcggactggcctcccaagcgtggggcgacaagctgccggagctgcaatgggccgcggctggggattcttgtttggcctcctgggcgccgtgtggctgctcagctcgggccacggagaggagcagcccccggagacagcggcacagaggtgcttctgccaggttagtggttacttggatgattgtacctgtgatgttgaaaccattgatagatttaataactacaggcttttcccaagactacaaaaacttcttgaaagtgactactttaggtattacaaggtaaacctgaagaggccgtgtcctttctggaatgacatcagccagtgtggaagaagggactgtgctgtcaaaccatgtcaatctgatgaagttcctgatggaattaaatctgcgagctacaagtattctgaagaagccaataatctcattgaagaatgtgaacaagctgaacgacttggagcagtggatgaatctctgagtgaggaaacacagaaggctgttcttcagtggaccaagcatgatgattcttcagataacttctgtgaagctgatgacattcagtcccctgaagctgaatatgtagatttgcttcttaatcctgagcgctacactggttacaagggaccagatgcttggaaaatatggaatgtcatctacgaagaaaactgttttaagccacagacaattaaaagacctttaaatcctttggcttctggtcaagggacaagtgaagagaacactttttacagttggctagaaggtctctgtgtagaaaaaagagcattctacagacttatatctggcctacatgcaagcattaatgtgcatttgagtgcaagatatcttttacaagagacctggttagaaaagaaatggggacacaacattacagaatttcaacagcgatttgatggaattttgactgaaggagaaggtccaagaaggcttaagaacttgtattttctctacttaatagaactaagggctttatccaaagtgttaccattcttcgagcgcccagattttcaactctttactggaaataaaattcaggatgaggaaaacaaaatgttacttctggaaatacttcatgaaatcaagtcatttcctttgcattttgatgagaattcattttttgctggggataaaaaagaagcacacaaactaaaggaggactttcgactgcattttagaaatatttcaagaattatggattgtgttggttgttttaaatgtcgtctgtggggaaagcttcagactcagggtttgggcactgctctgaagatcttattttctgagaaattgatagcaaatatgccagaaagtggacctagttatgaattccatctaaccagacaagaaatagtatcattattcaacgcatttggaagaatttctacaagtgtgaaagaattagaaaacttcaggaacttgttacagaatattcattaaagaaaacaagctgatatgtgcctgtttctggacaatggaggcgaaagagtggaatttcattcaaaggcataatagcaatgacagtcttaagccaaacattttatataaagttgcttttgtaaaggagaattatattgttttaagtaaacacatttttaaaaattgtgttaagtctatgtataatactactgtgagtaaaagtaatactttaataatgtggtacaaattttaaagtttaatattgaataaaaggaggattatcaaattcatatatgataaaagtgaatgttctaagtctctcaaactagcgttttatgtaataatatgtaatataaataaaactatggtaaatgtgacaagcatttaataggaaaatgctaaggaggcctcataaatgacccataattaccaacgtagaatttttcagtacatttagggttgctggatttagcaaataaaaataaagattgcccagttagatttgaatttcagataaacaattagttttttaatattttacatggaatatttggaaaatacttatactaaaaaattatttgtttgaaattcacatttaactgggagtcttgtattttatctggcaatcctaaaatacattggtatgaaacaaatcacttttagaagtatattgctattttgattgggttgtttttgtgtgtagaaacgtacaataacaactcaaaggcacaggagatttctaaacattgtgaaaagttgaatagattatatatttattctcataatactttcactaatactaaataaaatttggggaacactttttatttttatataatttccaatttacagaaaagtttcaaaaatagtacaaagagctctcttacccagattcactaattgttcatacgtgctttatctttcatgctttctctgtacacacacacacacacacaaatttttcctcaatcatttgaaagtcagttataggcatcatgccccttaaaccctaaatacttcagtgtgtaatactgaataattactaaaaatgattttctcagaaaaaaaaactcccacaattctggaactataatactgtaagccttagaataaataatactttcaagttccaatctaaagttctttttgagttttgttgcccgttttatgcttgatgtgtatagtaatagggtaggctatttattttattaaaattttttttagagacaaggttttgctgtgttgcccaagctggaacttgaacgactgggctgaagtgatcttcccacctcagcctcccaagtagctgggaatacaggtgtctgccaccatacccagtttcatttttgttttttatacccgaagttcatttcctttgtctccctaaaactgaactgtaattttgggaggttttcattagtggaagctcttcatttataaagctatttgaaggggtttaggaatttatatcacatggtaattgtagagaaaaagaagctatatacctcaaaatcgtgccctctttacatatgtcttatcaggtataacatgttgaaatgtcacattagtagtaaagtggggtttatttatatagtggttaagaaatgtcagtttacactgctgtatacttcttcttctgtgtccctaaggcctggtacagtgccaagcacatacttggtatccaataaatatttgttggatgaaaaaaaaaaaaaaaaaaaa

SEQ ID NO: 6 - protein, 468 aa, Homo sapiens
MGRGWGFLFGLLGAVWLLSSGHGEEQPPETAAQRCFCQVSGYLDDCTCDVETIDRFNNYRLFPRLQKLLESDYFRYYKVNLKRPCPFWNDISQCGRRDCAVKPCQSDEVPDGIKSASYKYSEEANNLIEECEQAERLGAVDESLSEETQKAVLQWTKHDDSSDNFCEADDIQSPEAEYVDLLLNPERYTGYKGPDAWKIWNVIYEENCFKPQTIKRPLNPLASGQGTSEENTFYSWLEGLCVEKRAFYRLISGLHASINVHLSARYLLQETWLEKKWGHNITEFQQRFDGILTEGEGPRRLKNLYFLYLIELRALSKVLPFFERPDFQLFTGNKIQDEENKMLLLEILHEIKSFPLHFDENSFFAGDKKEAHKLKEDFRLHFRNISRIMDCVGCFKCRLWGKLQTQGLGTALKILFSEKLIANMPESGPSYEFHLTRQEIVSLFNAFGRISTSVKELENFRNLLQNIH

SEQ ID NO: 7 - DNA, 940 bp, Home sapiens
ctgtcagctgaggatccagccgaaagaggagccaggcactcaggccacctgagtctactcacctggacaactggaatctggcaccaattctaaaccactcagcttctccgagctcacaccccggagatcacctgaggacccgagccattgatggactcggacgagaccgggttcgagcactcaggactgtgggtttctgtgctggctggtcttctgctgggagcctgccaggcacaccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcactcccggagccacccggaatcctggccccccagccccccgatgtgggctcctcggaccctctgagcatggtgggaccttcccagggccgaagccccagctacgcttcctgaagccagaggctgtttactatgacatctcctctttatttattaggttatttatcttatttatttttttatttttcttacttgagataataaagagttccagaggagaaaaaaaaaaaaaaaaaaaaaaaaaaaaaaaaaaaaaaaaaaaaaaaaaaaaaaa

SEQ ID NO: 8 - protein, 209 aa, Homo sapiens
MDSDETGFEHSGLWVSVLAGLLLGACQAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 9 - DNA, 3419 bp, Homo sapiens
gagtcgctcaggagcaagctgtggcaagctggagggagggacacatcccgtgttccatccactccctcccttctcagcagtcctcgcctgttctcacgtgctcacaggcagttaggcagaagtgatccccgtggctctgccaaagacaagcctgttgggttgaaagaagaagaagaagaagaaaaaaaaactcaggcaaagtcacagcctcaaaattgttcactgaaagaagcgtgagtggagaagtgtgagaagatgaatggaccggtggatggcttgtgtgaccactctctaagtgaaggagtcttcatgttcacatcggagtctgtgggagagggacacccggataagatctgtgaccagatcagtgatgcagtgctggatgcccatctcaagcaagaccccaatgccaaggtggcctgtgagacagtgtgcaagaccggcatggtgctgctgtgtggtgagatcacctcaatggccatggtggactaccagcgggtggtgagggacaccatcaagcacatcggctacgatgactcagccaagggctttgacttcaagacttgcaacgtgctggtggctttggagcagcaatccccagatattgcccagtgcgtccatctggacagaaatgaggaggatgtgggggcaggagatcagggtttgatgttcggctatgctaccgacgagacagaggagtgcatgcccctcaccatcatccttgctcacaagctcaacgcccggatggcagacctcaggcgctccggcctcctcccctggctgcggcctgactctaagactcaggtgacagttcagtacatgcaggacaatggcgcagtcatccctgtgcgcatccacaccatcgtcatctctgtgcagcacaacgaagacatcacgctggaggagatgcgcagggccctgaaggagcaagtcatcagggccgtggtgccggccaagtacctggacgaagacaccgtctaccacctgcagcccagtgggcggtttgtcatcggaggtccccagggggatgcgggtgtcactggccgtaagattattgtggacacctatggcggctggggggctcatggtggtggggccttctctgggaaggactacaccaaggtagaccgctcagctgcatatgctgcccgctgggtggccaagtctctggtgaaagcagggctctgccggagagtgcttgtccaggtttcctatgccattggtgtggccgagccgctgtccatttccatcttcacctacggaacctctcagaagacagagcgagagctgctggatgtggtgcataagaacttcgacctccggccgggcgtcattgtcagggatttggacttgaagaagcccatctaccagaagacagcatgctacggccatttcggaagaagcgagttcccatgggaggttcccaggaagcttgtattttagagccagggggagctgggcctggtctcaccctggaggcacctggtggccatgctcctcttccccagacgcctggctgctgatcgccttccccacccaccaaccctcagggcaaagccaggtccctctcatttagcctgtcctgtcatcatcatggccagctggaggcaggggcttcctggtgctggaggttggatcttgatgtaaggatgggcatggtgttctcctgctgctccctcagactggggcaatgttaatttagtggaaaaggcacccccgtcaagagtgaattccctcactcgtctcccccaacagctggaccctgaccagctccccctccctccccttgcctgtgccaggtgaggtcagcacatctcaacaggcctcagggctccttgtgggcctgggctcctggacccccctttcacaggcagccagtgccctgagccagggtctccagaaagccccacccaggccaggcatgtggcaggggttagagcaggactgatgtctcctaagcacctgtaatgtgcgagggacccagctaataactgatctcgttttttcttcactgcaacatgatgaggtagtaccttttatatcccatttatagatgggggaaagcaaagcacagagagtctggataacttccacagggtcccacagccacgtgtttagacctagatgtataactaggagctttgactcaggagcctgtgacataccccctcccccaccgttgtctcatgccagtaacaggctcaaacaatgacaaagcagattcagaaatgaggccatggactctgtcctgaaggcctgaggttactggaaattaggggattaacccactagctcttgttgagccgtgggcaattgtctgaaaagtgaagacagaaccacagggctattttgtttgcttcatgtgtcccagaagatgactgagggtgagttggcttacctggcccatcagggtaggctggagttagggactgaccagcagctttagaatcccagccccctgaccactcagagacatgcagagattgggtttttggacttctggggtaagtggtctaagtccagtccagtcctatctgggcttcctggagcagaagcagcaacttgtcctagcacagatggccagccccttagacagaggccctcaagtctttctctttccctggtcccttgtatcccctgcaggctgagtgcatttggagggagtgagtggccctttcggatccagggaggctggtcctatggcctcatgttaaataggcggggcttgccttctggtgttggacaagcttctgagacgtcatgaggagattctgcctttgccaggtgactgtctggggagcgggtctgctcccaaggggcctgagcagtccttggcctgctaaggtcttggaacttgcctgcctttccatccatggccagcagcacctgccctacctgccccacttgtccttagcctggacctctgacagcagcatctctaccttctccccagctcccaggaccacaggctcaggcaggggcctccatgggccccaggggaacactggggacttggcctctctctagggtacatggtgctgggagaggcagcccaggaagtctcatctggggagcaggcagccagcatctgggccttggcctggagcacaaagaccctggctttcattttctctcaggtgaaaggaaattaaggcaacaaaagaagcccggctcctggtcacctaggaagcctcagattccttcccatggagggagggagtggtttgcaggtggccaagttcctctaacttggctcacactcgacatgaaaattcagaattttatactttccctaccctctagagaaataagatcttttttgtcagtttgtttgtatgaaactaaagcctttatttgttaatagttcctgctaaaacaatgaataaaaactcaaggagcaactaaaaaaaaaaaaaaaa

SEQ ID NO: 10 - protein, 337 aa, Homo sapiens
MNGPVDGLCDHSLSEGVFMFTSESVGEGHPDKICDQISDAVLDAHLKQDPNAKVACETVCKTGMVLLCGEITSMAMVDYQRVVRDTIKHIGYDDSAKGFDFKTCNVLVALEQQSPDIAQCVHLDRNEEDVGAGDQGLMFGYATDETEECMPLTIILAHKLNARMADLRRSGLLPWLRPDSKTQVTVQYMQDNGAVIPVRIHTIVISVQHNEDITLEEMRRALKEQVIRAVVPAKYLDEDTVYHLQPSGRFVIGGPQGDAGVTGRKIIVDTYGGWGAHGGGAFSGKDYTKVDRSAAYAARWVAKSLVKAGLCRRVLVQVSYAIGVAEPLSISIFTLVF

SEQ ID NO: 11 - DNA, 2169 bp, Homo sapiens
gtggcccgaagcgggggccgccccggtcgggcggggaggcggggaggtggccactggctccgccccgctcccctctgacccggtgtctagtctctccgtcttcctgttccaaaccacgtggacgcgtctgggctgctggaggcagcccgagccgccgccgtcggtgtcgccgccaccaccaccatcggagtcacgagtcccgcgtctgtccgaagtcgccgctctcgggctgctcacgtctcttcggagagcgcgcacatggcgactcaggcgcactccctcagctacgcagggtgcaacttcttgcgccaacgtctggtcctgtctaccctgagcgggcgccccgtcaaaatccgaaagattcgggccagagacgacaacccgggcctccgagattttgaagccagcttcataaggctattggacaaaataacgaatggttctcgaattgaaataaaccaaacaggaacaaccttatattatcagcctggcctcctgtatggtggatctgtggaacatgactgtagcgtccttcgtggcattgggtattacctggagagtcttctttgcttggctccatttatgaagcacccgttaaaaatagttctacgaggagtgaccaatgatcaggttgacccttcagttgatgttcttaaggcaacagcactccctttgttgaaacaatttgggattgatggtgaatcatttgaactgaagattgtgcgacggggaatgcctcccggaggaggaggcgaagtggttttctcatgtcctgtgaggaaggtcttgaagcccattcaactcacagatccaggaaaaatcaaacgtattagaggaatggcgtactctgtacgtgtgtcacctcagatggcgaaccggattgtggattctgcaaggagcatcctcaacaagttcatacctgatatctatatttacacagatcacatgaaaggagtcaactctgggaagtctccgggctttgggttgtcactggttgctgagaccaccagtggcaccttcctcagtgctgaactggcctccaacccccagggccagggagcagcagtacttccagaggaccttggcaggaactgtgcccggctgctgctggaggaaatctacaggggtggatgcgtagactcgaccaaccaaagcctggcgctactactcatgacccttggacagcaggatgtttccaaagtcctgctaggccctctctctccctacacgatagaatttttgcggcatttgaagagctttttccagattatgtttaaaattgaaaccaagccatgtggtgaagaactcaagggtggggataaagtgctgatgacctgtgttggcattggtttctccaaccttagcaagaccctcaagtgataaccatcacaagataaggccccaatgcctacagacaaagcagaagctgccacggacaccaatgggaccaagtccaaatggattaatccaggacagaatagccacttgcttaattttctgtgaagaaatatcaatatacaaataaaagacatccctgtagcatatggtttccagctgtttctccagtggcattgccattgcccaggaggggcccagtcaccatgagagctcccttgccttacctggaggaagaatgtgccttcaggccacagtcgtgctgctagaacagtctcgtagctgcagttcagctgtgcttcctcagcctactatcataggcttcctcagccctctgtcatatggctgttttgcaaacctgtggagtctgttactgttctttctgcaaggactcacctccttgagccttggtttttgttgtagggattaaatgagataatatgagtggcagctcttcatgagtcctgcagtgctaagcaaatgtcagaaattggtgtattagactatttatctttgatcttctgaatggattgctgtcatggacacggacacggatcttcatctggttcattgtatttatatgtgagggatggatggctgcggggctccaagtaagttattgggatgtttttatattccaggtgtgctgtacattcttattttattttcacaatagctctgtgatgtaagtgctatctccatgagaaaattcataaagggtgttttgttcctttgaaatgtataatgtaaagacattaaatctcctcatttaag

SEQ ID NO: 12 - protein, 373 aa, Homo sapiens
MATQAHSLSYAGCNFLRQRLVLSTLSGRPVKIRKIRARDDNPGLRDFEASFIRLLDKITNGSRIEINQTGTTLYYQPGLLYGGSVEHDCSVLRGIGYYLESLLCLAPFMKHPLKIVLRGVTNDQVDPSVDVLKATALPLLKQFGIDGESFELKIVRRGMPPGGGGEVVFSCPVRKVLKPIQLTDPGKIKRIRGMAYSVRVSPQMANRIVDSARSILNKFIPDIYIYTDHMKGVNSGKSPGFGLSLVAETTSGTFLSAELASNPQGQGAAVLPEDLGRNCARLLLEEIYRGGCVDSTNQSLALLLMTLGQQDVSKVLLGPLSPYTIEFLRHLKSFFQIMFKIETKPCGEELKGGDKVLMTCVGIGFSNLSKTLK

SEQ ID NO: 13 - DNA, 2596 bp, Homo sapiens
gtggggcgcgggcgcgcgcacggacgtccagcacgcacgcacgcacgtccagcacacacgcacatccagcacgcacgcacgtacgtccggcacttccggccgcggcggcctcagcgccggcccgaagggaccaggccgccgtccccagcgagaggcatgcagcgctgaggagcggcgacccagcacggcggcgccatgaacctcctgccgtgtaaccctcacggcaacgggctgctctacgccggcttcaaccaggaccacggatgctttgcgtgtgggatggaaaatggattccgagtctataacactgatccactaaaagaaaaagagaaacaagaatttctagaaggaggagttggccatgttgaaatgttatttcgctgcaactatttagctttagttggtggtggaaaaaagccgaaataccctcccaacaaagtaatgatctgggatgacctgaagaagaagactgttattgaaatagaattttctacagaagtcaaggcagtcaagctgcggcgagatagaattgtggtggttttggactccatgattaaggtgttcacattcacacacaatccccatcagttgcacgtcttcgaaacctgctataaccccaaaggcctctgtgtcctttgtcccaatagtaacaactccctcctggcctttccgggcacgcacacgggccatgtgcagcttgtggacctggccagcacggagaagccacccgtggacattcctgcacacgagggtgtcctgagctgcattgcactcaacctgcagggaacaagaattgcaactgcatccgagaaagggacgcttataagaatatttgatacttcatcagggcatttaatccaggaactgcgaagaggatctcaagcagccaatatttactgcatcaacttcaatcaggatgcgtccctcatctgcgtatccagcgaccacggcacagtgcatatttttgcagctgaagatccaaaaaggaataaacagtccagtttggcctcagccagtttccttccaaaatacttcagttccaagtggagtttctccaagtttcaggttccctcaggctctccgtgcatttgtgcctttggaacagagccaaacgccgtcattgcaatttgtgcagacggcagctactacaaattcctgttcaaccccaagggggagtgcatccgagatgtctacgcgcagtttctagagatgaccgatgacaagctgtgactccagctgggggcgccacagcacccaccacctgccgccttcagactctcggggctggtgccagtgccccaggggcctcctgggccacgggctggaggggctgcccagggaccttggtctcgaagccatacgtggttgtctgctttcctaaggactcccatttccagtattaaagagagaatcatcatcaaggcaccgtaggtaactcagtggctgtgaccagctcgactggcggccactggctgttcccatgagttcagctgtgacgttagcttcagtggctccgccgcatcctcacactgacgggggctccatacggacctggggactgggctgagagggtggacgagttcaggtttgtttttgcagcagattccgtcgttcttactgagtctgcagcgggggagtgaacaagtgtgcagatgtaagttcttacatgataagcagattgaatacaacaccagcagcttgccttagaaaaggagaaaggaattccttttcccgcccgaacatgaagaaaaatgacctgaccctgtagagagaacacagtgtgaatgtttcccctcgtgtgagcccagcctgtggtcttctccgtacccgcaacgtggtcatctgtgcccgtgacgtcacctgtgcccgtgcgtggcgtccccgtctccgttggggccattagaatgaggcagacaccaggccactctagaagccgagccgtcacacctcaggcgtgtgcggggcggggacggggggtctcctggttacattttggattaaacctgtttcccggttatgtgtagggaacagcagagtgatgcacgaactttgaacattcgttatggggaaaacatcctttaacttcggggtcgtctgccagagcagggtctgggagggtccatgcagttcccgctggtgtggagggaaatgccctggtctggcctccgagcccccaggtccaccgtctcccctcccctcatttgtaagaatagctacacactaacattttgggaaggagaggcacataactttttttaacatttggtaactaggttatgggctctacattgtcagctacttgggatatatatttaattttcttaaattcccgttaaactctattttatggttttgatttcagattgcaaacatgtaaaacctgcatagcagcgagttctcggttttgccggtttctttagttctttactgtcactgtcatgtaatcagctaattctctgtggatgttgctgtaaagtatgcatgttcctttcatgtgtatttaatcatgatgtttaattttgcacacttatttgtaatgtttcttttaaataaaagtgactaattttgttgtagtctggacctgtaaaaaaaaaaa

SEQ ID NO: 14 - protein, 344 aa, Homo sapiens
MNLLPCNPHGNGLLYAGFNQDHGCFACGMENGFRVYNTDPLKEKEKQEFLEGGVGHVEMLFRCNYLALVGGGKKPKYPPNKVMIWDDLKKKTVIEIEFSTEVKAVKLRRDRIVVVLDSMIKVFTFTHNPHQLHVFETCYNPKGLCVLCPNSNNSLLAFPGTHTGHVQLVDLASTEKPPVDIPAHEGVLSCIALNLQGTRIATASEKGTLIRIFDTSSGHLIQELRRGSQAANIYCINFNQDASLICVSSDHGTVHIFAAEDPKRNKQSSLASASFLPKYFSSKWSFSKFQVPSGSPCICAFGTEPNAVIAICADGSYYKFLFNPKGECIRDVYAQFLEMTDDKL